(19) United States
(12) Patent Application Publication
(10) Pub. No.: US 2005/0124022 A1
(43) Pub. Date: Jun. 9, 2005

(54) Novel sulfurylase-luciferase fusion proteins and thermostable sulfurylase

(76) Inventors: Maithreyan Srinivasan, Hamden, CT (US); Michael Reifler, Hamden, CT (US)

Correspondence Address: MINTZ LEVIN COHN FERRIS GLOVSKY & POPEO, 666 THIRD AVENUE, NEW YORK, NY, 10017, US

(21) Appl. No.: 10/494,073

(22) PCT Filed: Oct. 30, 2002
(86) PCT No.: PCT/US02/34612

Related U.S. Application Data
(63) Continuation-in-part of application No. 10/122,706, filed on Apr. 11, 2002.
Application No. PCT/US02/34612 is a continuation-in-part of application No. 10/154,515, filed on May 23, 2002.
Application No. PCT/US02/34612 is a continuation-in-part of application No. 10/122,706, filed on Apr. 11, 2002.
(60) Provisional application No. 60/335,949, filed on Oct. 30, 2001.
(60) Provisional application No. 60/335,949, filed on Oct. 30, 2001.
(60) Provisional application No. 60/349,076, filed on Jan. 16, 2002.
(60) Provisional application No. 60/335,949, filed on Oct. 30, 2001.
(60) Provisional application No. 60/349,076, filed on Jan. 16, 2002.

(51) Int. Cl. C12Q 1/42; C12N 9/10; C12N 9/12; C12N 1/21

(52) U.S. Cl. 435/21; 435/194; 435/193; 435/252.3

(57) ABSTRACT

The present invention relates to the field of DNA recombinant technology. More specifically, this invention relates to fusion proteins comprising an ATP generating polypeptide joined to a polypeptide that converts ATP into a detectable entity. Accordingly, this invention focuses on sulfurylase-luciferase fusion proteins. This invention also relates to pharmaceutical compositions containing the fusion proteins and methods for using them.

### FIELD OF THE INVENTION

The invention relates generally to fusion proteins that are useful as reporter proteins, in particular to fusion proteins of ATP sulfurylase and luciferase which are utilized to achieve an efficient conversion of pyrophosphate (PPi) to light. This invention also relates to a novel thermostable sulfurylase which can be used in the detection of inorganic pyrophosphate, particularly in the sequencing of nucleic acid.

### BACKGROUND OF THE INVENTION

ATP sulfurylase has been identified as being involved in sulfur metabolism. It catalyzes the initial reaction in the metabolism of inorganic sulfate (SO₄⁻²); see e.g. Robbins and Lipmann, 1958. J. Biol. Chem. 233: 686-690; Hawes and Nicholas, 1973. Biochem. J. 133: 541-550). In this reaction SO₄⁻² is activated to adenosine 5′-phosphosulfate (APS). ATP sulfurylase is also commonly used in pyrophosphate sequencing methods. In order to convert pyrophosphate (PPi) generated from the addition of dNMP to a growing DNA chain to light, PPi must first be converted to ATP by ATP sulfurylase.

ATP produced by an ATP sulfurylase can also be hydrolyzed using enzymatic reactions to generate light. Light-emitting chemical reactions (i.e., chemiluminescence) and biological reactions (i.e., bioluminescence) are widely used in analytical biochemistry for sensitive measurements of various metabolites. In bioluminescent reactions, the chemical reaction that leads to the emission of light is enzyme-catalyzed. For example, the luciferin-luciferase system allows for specific assay of ATP. Thus, both ATP generating enzymes, such as ATP sulfurylase, and light emitting enzymes, such as luciferase, could be useful in a number of different assays for the detection and/or concentration of specific substances in fluids and gases. Since high physical and chemical stability is sometimes required for enzymes involved in sequencing reactions, a thermostable enzyme is desirable.

Because the product of the sulfurylase reaction is consumed by luciferase, proximity between these two enzymes by covalently linking the two enzymes in the form of a fusion protein would provide for a more efficient use of the substrate. Substrate channeling is a phenomenon in which substrates are efficiently delivered from enzyme to enzyme without equilibration with other pools of the same substrates. In effect, this creates local pools of metabolites at high concentrations relative to those found in other areas of the cell. Therefore, a fusion of an ATP generating polypeptide and an ATP converting peptide could benefit from the phenomenon of substrate channeling and would reduce production costs and increase the number of enzymatic reactions that occur during a given time period.

All patents and publications cited throughout the specification are hereby incorporated by reference into this specification in their entirety in order to more fully describe the state of the art to which this invention pertains.

### SUMMARY OF THE INVENTION

The invention provides a fusion protein comprising an ATP generating polypeptide bound to a polypeptide which converts ATP into an entity which is detectable. In one aspect, the invention provides a fusion protein comprising a sulfurylase polypeptide bound to a luciferase polypeptide. This invention provides a nucleic acid that comprises an open reading frame that encodes a novel thermostable sulfurylase polypeptide. In a further aspect, the invention provides for a fusion protein comprising a thermostable sulfurylase joined to at least one affinity tag.

In another aspect, the invention provides a recombinant polynucleotide that comprises a coding sequence for a fusion protein having a sulfurylase poylpeptide sequence joined to a luciferase polypeptide sequence. In a further aspect, the invention provides an expression vector for expressing a fusion protein. The expression vector comprises a coding sequence for a fusion protein having: (i) a regulatory sequence, (ii) a first polypeptide sequence of an ATP generating polypeptide and (iii) a second polypeptide sequence that converts ATP to an entity which is detectable. In an additional embodiment, the fusion protein comprises a sulfurylase polypeptide and a luciferase polypeptide. In another aspect, the invention provides a transformed host cell which comprises the expression vector. In an additional aspect, the invention provides a fusion protein bound to a mobile support. The invention also includes a kit comprising a sulfurylase-luciferase fusion protein expression vector.

The invention also includes a method for determining the nucleic acid sequence in a template nucleic acid polymer, comprising: (a) introducing the template nucleic acid polymer into a polymerization environment in which the nucleic acid polymer will act as a template polymer for the synthesis of a complementary nucleic acid polymer when nucleotides are added; (b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released; (c) separately recovering each of the feedstocks from the polymerization environment; and (d) measuring the amount of PPi with an ATP generating polypeptide-ATP converting polypeptide fusion protein in each of the recovered feedstocks to determine the identity of each nucleotide in the complementary polymer and thus the sequence of the template polymer. In one embodiment, the amount of inorganic pyrophosphate is measured by the steps of: (a) adding adenosine-5′-phosphosulfate to the feedstock; (b) combining the recovered feedstock containing adenosine-5′-phosphosulfate with an ATP generating polypeptide-ATP converting polypeptide fusion protein such that any inorganic pyrophosphate in the recovered feedstock and the adenosine-5′-phosphosulfate will react to the form ATP and sulfate; (c) combining the ATP and sulfate-containing feedstock with luciferin in the presence of oxygen such that the ATP is consumed to produced AMP, inorganic pyrophosphate, carbon dioxide and light; and (d) measuring the amount of light produced.

In another aspect, the invention includes a method wherein each feedstock comprises adenosine-5′-phosphosulfate and luciferin in addition to the selected nucleotide base, and the amount of inorganic pyrophosphate is determined by reacting the inorganic pyrophosphate feedstock with an ATP generating polypeptide-ATP converting polypeptide fusion protein thereby producing light in an amount proportional to the amount of inorganic pyrophosphate, and measuring the amount of light produced.

In another aspect, the invention provides a method for sequencing a nucleic acid, the method comprising: (a) providing one or more nucleic acid anchor primers; (b) providing a plurality of single-stranded circular nucleic acid templates disposed within a plurality of cavities on a planar surface, each cavity forming an analyte reaction chamber, wherein the reaction chambers have a center to center spacing of between 5 to 200 μm; (c) annealing an effective amount of the nucleic acid anchor primer to at least one of the single-stranded circular templates to yield a primed anchor primer-circular template complex; (d) combining the primed anchor primer-circular template complex with a polymerase to form an extended anchor primer covalently linked to multiple copies of a nucleic acid complementary to the circular nucleic acid template; (e) annealing an effective amount of a sequencing primer to one or more copies of said covalently linked complementary nucleic acid; (f) extending the sequencing primer with a polymerase and a predetermined nucleotide triphosphate to yield a sequencing product and, if the predetermined nucleotide triphosphate is incorporated onto the 3′ end of said sequencing primer, a sequencing reaction byproduct; and (g) identifying the sequencing reaction byproduct with the use of a ATP generating polypeptide-ATP converting polypeptide fusion protein, thereby determining the sequence of the nucleic acid.

In one aspect, the invention provides a method for sequencing a nucleic acid, the method comprising: (a) providing at least one nucleic acid anchor primer; (b) providing a plurality of single-stranded circular nucleic acid templates in an array having at least 400,000 discrete reaction sites; (c) annealing a first amount of the nucleic acid anchor primer to at least one of the single-stranded circular templates to yield a primed anchor primer-circular template complex; (d) combining the primed anchor primer-circular template complex with a polymerase to form an extended anchor primer covalently linked to multiple copies of a nucleic acid complementary to the circular nucleic acid template; (e) annealing a second amount of a sequencing primer to one or more copies of the covalently linked complementary nucleic acid; (f) extending the sequencing primer with a polymerase and a predetermined nucleotide triphosphate to yield a sequencing product and, when the predetermined nucleotide triphosphate is incorporated onto the 3′ end of the sequencing primer, to yield a sequencing reaction byproduct; and (g) identifying the sequencing reaction byproduct with the use of a ATP generating polypeptide-ATP converting polypeptide fusion protein, thereby determining the sequence of the nucleic acid at each reaction site that contains a nucleic acid template.

In another aspect, the invention includes a method of determining the base sequence of a plurality of nucleotides on an array, the method comprising the steps of: (a) providing a plurality of sample DNAs, each disposed within a plurality of cavities on a planar surface, each cavity forming an analyte reaction chamber, wherein the reaction chambers have a center to center spacing of between 5 to 200 μm, (b) adding an activated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates; (c) determining whether or not the nucleoside 5′-triphosphate precursor was incorporated into the primer strands through detection of a sequencing byproduct with a ATP generating polypeptide-ATP converting polypeptide fusion protein, thus indicating that the unpaired nucleotide residue of the template has a nitrogenous base composition that is complementary to that of the incorporated nucleoside 5′-triphosphate precursor; and (d) sequentially repeating steps (b) and (c), wherein each sequential repetition adds and, detects the incorporation of one type of activated nucleoside 5′-triphosphate precursor of known nitrogenous base composition; and

(e) determining the base sequence of the unpaired nucleotide residues of the template in each reaction chamber from the sequence of incorporation of said nucleoside precursors.

In one aspect, the invention includes a method for determining the nucleic acid sequence in a template nucleic acid polymer, comprising: (a) introducing a plurality of template nucleic acid polymers into a plurality of cavities on a planar surface, each cavity forming an analyte reaction chamber, wherein the reaction chambers have a center to center spacing of between 5 to 200 μm, each reaction chamber having a polymerization environment in which the nucleic acid polymer will act as a template polymer for the synthesis of a complementary nucleic acid polymer when nucleotides are added; (b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released; (c) detecting the formation of inorganic pyrophosphate with an ATP generating polypeptide-ATP converting polypeptide fusion protein to determine the identify of each nucleotide in the complementary polymer and thus the sequence of the template polymer.

In one aspect, the invention provides a method of identifying the base in a target position in a DNA sequence of sample DNA including the steps comprising: (a) disposing sample DNA within a plurality of cavities on a planar surface, each cavity forming an analyte reaction chamber, wherein the reaction chambers have a center to center spacing of between 5 to 200 μm, said DNA being rendered single stranded either before or after being disposed in the reaction chambers, (b) providing an extension primer which hybridizes to said immobilized single-stranded DNA at a position immediately adjacent to said target position; (c) subjecting said immobilized single-stranded DNA to a polymerase reaction in the presence of a predetermined nucleotide triphosphate, wherein if the predetermined nucleotide triphosphate is incorporated onto the 3′ end of said sequencing primer then a sequencing reaction byproduct is formed; and (d) identifying the sequencing reaction byproduct with a ATP generating polypeptide-ATP converting polypeptide fusion protein, thereby determining the nucleotide complementary to the base at said target position.

The invention also includes a method of identifying a base at a target position in a sample DNA sequence comprising: (a) providing sample DNA disposed within a plurality of cavities on a planar surface, each cavity forming an analyte reaction chamber, wherein the reaction chambers have a center to center spacing of between 5 to 200 μm, said DNA being rendered single stranded either before or after being disposed in the reaction chambers; (b) providing an extension primer which hybridizes to the sample DNA immediately adjacent to the target position; (c) subjecting the sample DNA sequence and the extension primer to a polymerase reaction in the presence of a nucleotide triphosphate whereby the nucleotide triphosphate will only become incorporated and release pyrophosphate (PPi) if it is complementary to the base in the target position, said nucleotide triphosphate being added either to separate aliquots of sample-primer mixture or successively to the same sample-primer mixture; (d) detecting the release of PPi with an ATP generating polypeptide-ATP converting polypeptide fusion protein to indicate which nucleotide is incorporated.

In one aspect, the invention provides a method of identifying a base at a target position in a single-stranded sample DNA sequence, the method comprising: (a) providing an extension primer which hybridizes to sample DNA immediately adjacent to the target position, said sample DNA disposed within a plurality of cavities on a planar surface, each cavity forming an analyte reaction chamber, wherein the reaction chambers have a center to center spacing of between 5 to 200 um, said DNA being rendered single stranded either before or after being disposed in the reaction chambers; (b) subjecting the sample DNA and extension primer to a polymerase reaction in the presence of a predetermined deoxynucleotide or dideoxynucleotide whereby the deoxynucleotide or dideoxynucleotide will only become incorporated and release pyrophosphate (PPi) if it is complementary to the base in the target position, said predetermined deoxynucleotides or dideoxynucleotides being added either to separate aliquots of sample-primer mixture or successively to the same sample-primer mixture, (c) detecting any release of PPi with an ATP generating polypeptide-ATP converting polypeptide fusion protein to indicate which deoxynucleotide or dideoxynucleotide is incorporated; characterized in that, the PPi-detection enzyme(s) are included in the polymerase reaction step and in that in place of deoxy- or dideoxy adenosine triphosphate (ATP) a dATP or ddATP analogue is used which is capable of acting as a substrate for a polymerase but incapable of acting as a substrate for a said PPi—detection enzyme.

In another aspect, the invention includes a method of determining the base sequence of a plurality of nucleotides on an array, the method comprising: (a) providing a plurality of sample DNAs, each disposed within a plurality of cavities on a planar surface, each cavity forming an analyte reaction chamber, wherein the reaction chambers have a center to center spacing of between 5 to 200 μm, (b) converting PPi into light with an ATP generating polypeptide-ATP converting polypeptide fusion protein; (c) detecting the light level emitted from a plurality of reaction sites on respective portions of an optically sensitive device; (d) converting the light impinging upon each of said portions of said optically sensitive device into an electrical signal which is distinguishable from the signals from all of said other regions; (e) determining a light intensity for each of said discrete regions from the corresponding electrical signal; (f) recording the variations of said electrical signals with time.

In one aspect, the invention provides a method for sequencing a nucleic acid, the method comprising: (a) providing one or more nucleic acid anchor primers; (b) providing a plurality of single-stranded circular nucleic acid templates disposed within a plurality of cavities on a planar surface, each cavity forming an analyte reaction chamber, wherein the reaction chambers have a center to center spacing of between 5 to 200 μm; (c) converting PPi into a detectable entity with the use of an ATP generating polypeptide-ATP converting polypeptide fusion protein; (d) detecting the light level emitted from a plurality of reaction sites on respective portions of an optically sensitive device; (e) converting the light impinging upon each of said portions of said optically sensitive device into an electrical signal which is distinguishable from the signals from all of said other regions; (f) determining a light intensity for each of said discrete regions from the corresponding electrical signal; (g) recording the variations of said electrical signals with time.

In another aspect, the invention includes a method for sequencing a nucleic acid, the method comprising: (a) providing at least one nucleic acid anchor primer; (b) providing a plurality of single-stranded circular nucleic acid templates in an array having at least 400,000 discrete reaction sites; (c) converting PPi into a detectable entity with an ATP generating polypeptide-ATP converting polypeptide fusion protein; (d) detecting the light level emitted from a plurality of reaction sites on respective portions of an optically sensitive device; (e) converting the light impinging upon each of said portions of said optically sensitive device into an electrical signal which is distinguishable from the signals from all of said other regions; (f) determining a light intensity for each of said discrete regions from the corresponding electrical signal; (g) recording the variations of said electrical signals with time.

In another aspect, the invention includes an isolated polypeptide comprising an amino acid sequence selected from the group consisting of: (a) a mature form of an amino acid sequence of SEQ ID NO: 2; (b) a variant of a mature form of an amino acid sequence of SEQ ID NO: 2; an amino acid sequence of SEQ ID NO: 2; (c) a variant of an amino acid sequence of SEQ ID NO: 2, wherein one or more amino acid residues in said variant differs from the amino acid sequence of said mature form, provided that said variant differs in no more than 5% of amino acid residues from said amino acid sequence; (d) and at least one conservative amino acid substitution to the amino acid sequences in (a), (b), (c) or (d). The invention also includes an antibody that binds immunospecifically to the polypeptide of (a), (b), (c) or (d).

In another aspect, the invention includes an isolated nucleic acid molecule comprising a nucleic acid sequence encoding a polypeptide comprising an amino acid sequence selected from the group consisting of: (a) a mature form of an amino acid sequence of SEQ ID NO: 2; (b) a variant of a mature form of an amino acid sequence of SEQ ID NO: 2, wherein one or more amino acid residues in said variant differs from the amino acid sequence of said mature form, provided that said variant differs in no more than 5% of the amino acid residues from the amino acid sequence of said mature form; (c) an amino acid sequence of SEQ ID NO: 2; (d) a variant of an amino acid sequence of SEQ ID NO: 2, wherein one or more amino acid residues in said variant differs from the amino acid sequence of said mature form, provided that said variant differs in no more than 15% of amino acid residues from said amino acid sequence; a nucleic acid fragment encoding at least a portion of a polypeptide comprising an amino acid sequence of SEQ ID NO: 2, or a variant of said polypeptide, wherein one or more amino acid residues in said variant differs from the amino acid sequence of said mature form, provided that said variant differs in no more than 5% of amino acid residues from said amino acid sequence; (e) and a nucleic acid molecule comprising the complement of (a), (b), (c), (d) or (e). In a further aspect, the invention provides a nucleic acid molecule wherein the nucleic acid molecule comprises nucleotide sequence selected from the group consisting of: (a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence; an isolated second polynucleotide that is a complement of the first polynucleotide; (b) and a nucleic acid fragment of (a) or (b). The invention also includes a vector comprising the nucleic acid molecule of (a) or (b). In another aspect, the invention includes a cell comprising the vector.

In a further aspect, the invention includes a method for determining the nucleic acid sequence in a template nucleic acid polymer, comprising: (a) introducing the template nucleic acid polymer into a polymerization environment in which the nucleic acid polymer will act as a template polymer for the synthesis of a complementary nucleic acid polymer when nucleotides are added; (b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released; (c) separately recovering each of the feedstocks from the polymerization environment; and (d) measuring the amount of PPi with an ATP sulfurylase and a luciferase in each of the recovered feedstocks to determine the identity of each nucleotide in the complementary polymer and thus the sequence of the template polymer.

In another aspect, the invention provides a method for sequencing a nucleic acid, the method comprising: (a) providing one or more nucleic acid anchor primers; (b) providing a plurality of single-stranded circular nucleic acid templates disposed within a plurality of cavities in an array on a planar surface, each cavity forming an analyte reaction chamber, wherein the reaction chambers have a center to center spacing of between 5 to 200 μm and at least 400,000 discrete sites; (c) annealing an effective amount of the nucleic acid anchor primer to at least one of the single-stranded circular templates to yield a primed anchor primer-circular template complex; (d) combining the primed anchor primer-circular template complex with a polymerase to form an extended anchor primer covalently linked to multiple copies of a nucleic acid complementary to the circular nucleic acid template; (e) annealing an effective amount of a sequencing primer to one or more copies of said covalently linked complementary nucleic acid; (f) extending the sequencing primer with a polymerase and a predetermined nucleotide triphosphate to yield a sequencing product and, if the predetermined nucleotide triphosphate is incorporated onto the 3′ end of said sequencing primer, a sequencing reaction byproduct; and (g) identifying the sequencing reaction byproduct with the use of an ATP sulfurylase and a luciferase, thereby determining the sequence of the nucleic acid.

In another aspect, the invention provides a method for sequencing a nucleic acid, the method comprising: (a) providing at least one nucleic acid anchor primer; (b) providing a plurality of single-stranded circular nucleic acid templates in an array having at least 400,000 discrete reaction sites; (c) annealing a first amount of the nucleic acid anchor primer to at least one of the single-stranded circular templates to yield a primed anchor primer-circular template complex; (d) combining the primed anchor primer-circular template complex with a polymerase to form an extended anchor primer covalently linked to multiple copies of a nucleic acid complementary to the circular nucleic acid template; (e) annealing a second amount of a sequencing primer to one or more copies of the covalently linked complementary nucleic acid; (f) extending the sequencing primer with a polymerase and a predetermined nucleotide triphosphate to yield a sequencing product and, when the predetermined nucleotide triphosphate is incorporated onto the 3′ end of the sequencing primer, to yield a sequencing reaction byproduct; and (g) identifying the sequencing reaction byproduct with the use of a thermostable sulfurylase and a luciferase, thereby determining the sequence of the nucleic acid at each reaction site that contains a nucleic acid template.

In a further aspect, the invention includes a method of determining the base sequence of a plurality of nucleotides on an array, the method comprising: (a) providing a plurality of sample DNAs, each disposed within a plurality of cavities on a planar surface, each cavity forming an analyte reaction chamber, wherein the reaction chambers have a center to center spacing of between 5 to 200 μm, (b) adding an activated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates; (c) detecting whether or not the nucleoside 5′-triphosphate precursor was incorporated into the primer strands through detection of a sequencing byproduct with a thermostable sulfurylase and luciferase, thus indicating that the unpaired nucleotide residue of the template has a nitrogenous base composition that is complementary to that of the incorporated nucleoside 5′-triphosphate precursor; and (d) sequentially repeating steps (b) and (c), wherein each sequential repetition adds and, detects the incorporation of one type of activated nucleoside 5′-triphosphate precursor of known nitrogenous base composition; and (e) determining the base sequence of the unpaired nucleotide residues of the template in each reaction chamber from the sequence of incorporation of said nucleoside precursors.

### BRIEF DESCRIPTION OF THE DRAWINGS

FIG. 1 is one embodiment for a cloning strategy for obtaining the luciferase-sulfurylase sequence.

FIGS. 2A and 2B show the preparative agarose gel of luciferase and sulfurylase as well as sulfurylase-luciferase fusion genes.

FIG. 3 shows the results of experiments to determine the activity of the luciferase-sulfurylase fusion protein on NTA-agarose and MPG-SA solid supports.

### DETAILED DESCRIPTION OF THE INVENTION

This invention provides a fusion protein containing an ATP generating polypeptide bound to a polypeptide which converts ATP into an entity which is detectable. As used herein, the term “fusion protein” refers to a chimeric protein containing an exogenous protein fragment joined to another exogenous protein fragment. The fusion protein could include an affinity tag to allow attachment of the protein to a solid support or to allow for purification of the recombinant fusion protein from the host cell or culture supernatant, or both.

In a preferred embodiment, the ATP generating polypeptide and ATP converting polypeptide are from a eukaryote or a prokaryote. The eukaryote could be an animal, plant, fungus or yeast. In some embodiments, the animal is a mammal, rodent, insect, worm, mollusk, reptile, bird and amphibian. Plant sources of the polypeptides include but are not limited to Arabidopsis thaliana, Brassica napus, Allium sativum, Amaranthus caudatus, Hevea brasiliensis, Hordeum vulgare, Lycopersicon esculentum, Nicotiana tabacum, Oryza sativum, Pisum sativum, Populus trichocarpa, Solanum tuberosum, Secale cereale, Sambucus nigra, Ulmus americana or Triticum aestivum. Examples of fungi include but are not limited to Penicillum chrysogenum, Stachybotrys chartarum, Aspergillus fumigatus, Podospora anserina and Trichoderma reesei. Examples of sources of yeast include but are not limited to Saccharomyces cerevisiae, Candida tropicalis, Candida lypolitica, Candida utilis, Kluyveromyces lactis, Schizosaccharomyces pombe, Yarrowia lipolytica, Candida spp., Pichia spp. and Hansenula spp..

The prokaryote source could be bacteria or archaea. In some embodiments, the bacteria is E. coli, B. subtilis, Streptococcus gordonii, flavobacteria or green sulfur bacteria. In other embodiments, the archaea is Sulfolobus, Thermococcus, Methanobacterium, Halococcus, Halobacterium or Methanococcus jannaschii.

The ATP generating polypeptide can be a ATP sulfurylase, hydrolase or an ATP synthase. In a preferred embodiment, the ATP generating polypeptide is ATP sulfurylase. In one embodiment, the ATP sulfurylase is a thermostable sulfurylase cloned from Bacillus stearothermophilus (Bst) and comprising the nucleotide sequence of SEQ ID NO:1. This putative gene was cloned using genomic DNA acquired from ATCC (Cat. No. 12980D). The gene is shown to code for a functional ATP sulfurylase that can be expressed as a fusion protein with an affinity tag. The disclosed Bst sulfurylase nucleic acid (SEQ ID NO:1) includes the 1247 nucleotide sequence. An open reading frame (ORF) for the mature protein was identified beginning with an ATG codon at nucleotides 1-3 and ending with a TAA codon at nucleotides 1159-1161. The start and stop codons of the open reading frame are highlighted in bold type. The putative untranslated regions are underlined and found upstream of the initiation codon and downstream from the termination codon.

Bst Thermostable Sulfurylase Nucleotide Sequence

| | | |
|---|---|---|
| GTTATGAACATGAGTTTGAGCATTCCGCATGGCGGCACATTGATCAACCGTTGGAATCCG | 60 | (SEQ ID NO:1) |
| GATTACCCAATCGATGAAGCAACGAAAACGATCGAGCTGTCCAAAGCCGAACTAAGCGAC | 120 | |
| CTTGAGCTGATCGGCACAGGCGCCTACAGCCCGCTCACCGGGTTTTTAACGAAAGCCGAT | 180 | |
| TACGATGCGGTCGTAGAAACGATGCGCCTCGCTGATGGCACTGTCTGGAGCATTCCGATC | 240 | |
| ACGCTGGCGGTGACGGAAGAAAAAGCGAGTGAACTCACTGTCGGCGACAAAGCGAAACTC | 300 | |
| GTTTATGGCGGCGACGTCTACGGCGTCATTGAAATCGCCGATATTTACCGCCCGGATAAA | 360 | |
| ACGAAAGAAGCCAAGCTCGTCTATAAAACCGATGAACTCGCTCACCCGGGCGTGCGCAAG | 420 | |
| CTGTTTGAAAAACCAGATGTGTACGTCGGCGGAGCGGTTACGCTCGTCAAACGGACCGAC | 480 | |
| AAAGGCCAGTTTGCTCCGTTTTATTTCGATCCGGCCGAAACGCGGAAACGATTTGCCGAA | 540 | |
| CTCGGCTGGAATACCGTCGTCGGCTTCCAAACACGCAACCCGGTTCACCGCGCCCATGAA | 600 | |
| TACATTCAAAAATGCGCGCTTGAAATCGTGGACGGCTTGTTTTTAAACCCGCTCGTCGGC | 660 | |
| GAAACGAAAGCGGACGATATTCCGGCCGACATCCGGATGGAAAGCTATCAAGTGCTGCTG | 720 | |
| GAAAACTATTATCCGAAAGACCGCGTTTTCTTGGGCGTCTTCCAAGCTGCGATGCGCTAT | 780 | |
| GCCGGTCCGCGCGAAGCGATTTTCCATGCCATGGTGCGGAAAAACTTCGGCTGCACGCAC | 840 | |
| TTCATCGTCGGCCGCGACCATGCGGGCGTCGGCAACTATTACGGCACGTATGATGCGCAA | 900 | |
| AAAATCTTCTCGAACTTTACAGCCGAAGAGCTTGGCATTACACCGCTCTTTTTCGAACAC | 960 | |
| AGCTTTTATTGCACGAAATGCGAAGGCATGGCATCGACGAAAACATGCCCGCACGACGCA | 1020 | |
| CAATATCACGTTGTCCTTTCTGGCACGAAAGTCCGTGAAATGTTGCGTAACGGCCAAGTG | 1080 | |
| CCGCCGAGCACATTCAGCCGTCCGGAAGTGGCCGCCGTTTTGATCAAAGGGCTGCAAGAA | 1140 | |
| CGCGAAACGGTCACCCCGTCGACACGCTAAAGGAGGAGCGAGATGAGCACGAATATCGTT | 1200 | |
| TGGCATCATACATCGGTGACAAAAGAAGATCGCCGCCAACGCAACGG | 1247 | |

The Bst sulfurylase polypeptide (SEQ ID NO:2) is 386 amino acid residues in length and is presented using the three letter amino acid code.

Bst Sulfurylase Amino Acid Sequence

| Met Ser Leu Ser Ile Pro His Gly Gly Thr Leu Ile Asn Arg Trp Asn | (SEQ ID NO:2) |
|---|---|
| 1 5 10 15 | |
| Pro Asp Tyr Pro Ile Asp Glu Ala Thr Lys Thr Ile Glu Leu Ser Lys | |
| 20 25 30 | |
| Ala Glu Leu Ser Asp Leu Glu Leu Ile Gly Thr Gly Ala Tyr Ser Pro | |
| 35 40 45 | |
| Leu Thr Gly Phe Leu Thr Lys Ala Asp Tyr Asp Ala Val Val Glu Thr | |
| 50 55 60 | |
| Met Arg Leu Ala Asp Gly Thr Val Trp Ser Ile Pro Ile Thr Leu Ala | |
| 65 70 75 | |
| Val Thr Glu Glu Lys Ala Ser Glu Leu Thr Val Gly Asp Lys Ala Lys | |
| 80 85 90 95 | |
| Leu Val Tyr Gly Gly Asp Val Tyr Gly Val Ile Glu Ile Ala Asp Ile | |
| 100 105 110 | |
| Tyr Arg Pro Asp Lys Thr Lys Glu Ala Lys Leu Val Tyr Lys Thr Asp | |
| 115 120 125 | |
| Glu Leu Ala His Pro Gly Val Arg Lys Leu Phe Glu Lys Pro Asp Val | |
| 130 135 140 | |
| Tyr Val Gly Gly Ala Val Thr Leu Val Lys Arg Thr Asp Lys Gly Gln | |
| 145 150 155 | |
| Phe Ala Pro Phe Tyr Phe Asp Pro Ala Glu Thr Arg Lys Arg Phe Ala | |
| 160 165 170 175 | |
| Glu Leu Gly Trp Asn Thr Val Val Gly Phe Gln Thr Arg Asn Pro Val | |
| 180 185 190 | |
| His Arg Ala His Glu Tyr Ile Gln Lys Cys Ala Leu Glu Ile Val Asp | |
| 195 200 205 | |
| Gly Leu Phe Leu Asn Pro Leu Val Gly Glu Thr Lys Ala Asp Asp Ile | |
| 210 215 220 | |
| Pro Ala Asp Ile Arg Met Glu Ser Tyr Gln Val Leu Leu Glu Asn Tyr | |
| 225 230 235 | |
| Tyr Pro Lys Asp Arg Val Phe Leu Gly Val Phe Gln Ala Ala Met Arg | |
| 240 245 250 255 | |
| Tyr Ala Gly Pro Arg Glu Ala Ile Phe His Ala Met Val Arg Lys Asn | |
| 260 265 270 | |
| Phe Gly Cys Thr His Phe Ile Val Gly Arg Asp His Ala Gly Val Gly | |
| 275 280 285 | |
| Asn Tyr Tyr Gly Thr Tyr Asp Ala Gln Lys Ile Phe Ser Asn Phe Thr | |
| 290 295 300 | |
| Ala Glu Glu Leu Gly Ile Thr Pro Leu Phe Phe Glu His Ser Phe Tyr | |
| 305 310 315 | |
| Cys Thr Lys Cys Glu Gly Met Ala Ser Thr Lys Thr Cys Pro His Asp | |
| 320 325 330 335 | |
| Ala Gln Tyr His Val Val Leu Ser Gly Thr Lys Val Arg Glu Met Leu | |
| 340 345 350 | |
| Arg Asn Gly Gln Val Pro Pro Ser Thr Phe Ser Arg Pro Glu Val Ala | |
| 355 360 365 | |
| Ala Val Leu Ile Lys Gly Leu Gln Glu Arg Glu Thr Val Thr Pro Ser | |
| 370 375 380 | |
| Thr Arg | |
| 385 | |

In one embodiment, the thermostable sulfurylase is active at temperatures above ambient to at least 50° C. This property is beneficial so that the sulfurylase will not be denatured at higher temperatures commonly utilized in polymerase chain reaction (PCR) reactions or sequencing reactions. In one embodiment, the ATP sulfurylase is from a thermophile. The thermostable sulfurylase can come from thermophilic bacteria, including but not limited to, Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

The homology of twelve ATP sulfurylases can be shown graphically in the ClustalW analysis in Table 1. The alignment is of ATP sulfurylases from the following species: Bacillus stearothermophilus (Bst), University of Oklahoma—Strain 10 (Univ of OK), Aquifex aeolicus (Aae), Pyrococcus furiosus (Pfu), Sulfolobus solfataricus (Sso), Pyrobaculum aerophilum (Pae), Archaeoglobus fulgidus (Afu), Penicillium chrysogenum (Pch), Aeropyrum pernix (Ape), Saccharomyces cerevisiae (Sce), and Thermomonospora fusca (Tfu).

[image]

[image]

[image]

A thermostable sulfurylase polypeptide is encoded by the open reading frame (“ORF”) of a thermostable sulfurylase nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The invention further encompasses nucleic acid molecules that differ from the nucleotide sequences shown in SEQ ID NO:1 due to degeneracy of the genetic code and thus encode the same thermostable sulfurylase proteins as that encoded by the nucleotide sequences shown in SEQ ID NO:1. In another embodiment, an isolated nucleic acid molecule of the invention has a nucleotide sequence encoding a protein having an amino acid sequence shown in SEQ ID NO:2. In addition to the thermostable sulfurylase nucleotide sequence shown in SEQ ID NO:1 it will be appreciated by those skilled in the art that DNA sequence polymorphisms that lead to changes in the amino acid sequences of the thermostable sulfurylase polypeptides may exist within a population (e.g., the bacterial population). Such genetic polymorphism in the thermostable sulfurylase genes may exist among individuals within a population due to natural allelic variation. As used herein, the terms “gene” and “recombinant gene” refer to nucleic acid molecules comprising an open reading frame encoding a thermostable sulfurylase protein. Such natural allelic variations can typically result in 1-5% variance in the nucleotide sequence of the thermostable sulfurylase genes. Any and all such nucleotide variations and resulting amino acid polymorphisms in the thermostable sulfurylase polypeptides, which are the result of natural allelic variation and that do not alter the functional activity of the thermostable sulfurylase polypeptides, are intended to be within the scope of the invention.

Moreover, nucleic acid molecules encoding thermostable sulfurylase proteins from other species, and thus that have a nucleotide sequence that differs from the sequence SEQ ID NO:1 are intended to be within the scope of the invention. Nucleic acid molecules corresponding to natural allelic variants and homologues of the thermostable sulfurylase cDNAs of the invention can be isolated based on their homology to the thermostable sulfurylase nucleic acids disclosed herein using the human cDNAs, or a portion thereof, as a hybridization probe according to standard hybridization techniques under stringent hybridization conditions. The invention further includes the nucleic acid sequence of SEQ ID NO:1 and mature and variant forms thereof, wherein a first nucleotide sequence comprising a coding sequence differing by one or more nucleotide sequences from a coding sequence encoding said amino acid sequence, provided that no more than 11% of the nucleotides in the coding sequence differ from the coding sequence.

Another aspect of the invention pertains to nucleic acid molecules encoding a thermostable sulfurylase protein that contains changes in amino acid residues that are not essential for activity. Such thermostable sulfurylase proteins differ in amino acid sequence from SEQ ID NO:2 yet retain biological activity. In separate embodiments, the isolated nucleic acid molecule comprises a nucleotide sequence encoding a protein, wherein the protein comprises an amino acid sequence at least about 96%, 97%, 98% or 99% homologous to the amino acid sequence of SEQ ID NO:2. An isolated nucleic acid molecule encoding a thermostable sulfurylase protein homologous to the protein of SEQ ID NO: 2 can be created by introducing one or more nucleotide substitutions, additions or deletions into the nucleotide sequence of SEQ ID NO:1 such that one or more amino acid substitutions, additions or deletions are introduced into the encoded protein.

Mutations can be introduced into SEQ ID NO:2 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the thermostable sulfurylase protein is replaced with another amino acid residue from the same side chain family. Alternatively, in another embodiment, mutations can be introduced randomly along all or part of a thermostable sulfurylase coding sequence, such as by saturation mutagenesis, and the resultant mutants can be screened for thermostable sulfurylase biological activity to identify mutants that retain activity. Following mutagenesis of SEQ ID NO:1, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

The thermostable sulfurylase nucleic acid of the invention includes the nucleic acid whose sequence is provided herein, or fragments thereof. The invention also includes mutant or variant nucleic acids any of whose bases may be changed from the corresponding base shown herein while still encoding a protein that maintains its sulfurylase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

A thermostable sulfurylase nucleic acid can encode a mature thermostable sulfurylase polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated thermostable sulfurylase nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NO:1 or a complement of this aforementioned nucleotide sequence, can be isolated using standard molecular biology techniques and the sequence information provided herein. Using all or a portion of the nucleic acid sequence of SEQ ID NO:1 as a hybridization probe, thermostable sulfurylase molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to thermostable sulfurylase nucleotide sequences can be prepared by standard synthetic techniques, e.g., using an automated DNA synthesizer.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 89% identity over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of thermostable sulfurylase polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a thermostable sulfurylase polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. Homologous nucleic acid sequences include those nucleic acid sequences that encode conservative amino acid substitutions in SEQ ID NO:1, as well as a polypeptide possessing thermostable sulfurylase biological activity. Various biological activities of the thermostable sulfurylase proteins are described below.

The thermostable sulfurylase proteins of the invention include the sulfurylase protein whose sequence is provided herein. The invention also includes mutant or variant proteins any of whose residues may be changed from the corresponding residue shown herein while still encoding a protein that maintains its sulfurylase-like activities and physiological functions, or a functional fragment thereof. The invention further encompasses antibodies and antibody fragments, such as F_{ab} or (F_{ab})₂, that bind immunospecifically to any of the proteins of the invention. This invention also includes a variant or a mature form of the amino acid sequence of SEQ ID NO:2, wherein one or more amino acid residues in the variant differs in no more than 4% of the amino acic residues from the amino acid sequence of the mature form.

Several assays have been developed for detection of the forward ATP sulfurylase reaction. The colorimetric molybdolysis assay is based on phosphate detection (see e.g., Wilson and Bandurski, 1958. J. Biol. Chem. 233: 975-981), whereas the continuous spectrophotometric molybdolysis assay is based upon the detection of NADH oxidation (see e.g., Seubert, et al, 1983. Arch. Biochem. Biophys. 225: 679-691; Seubert, et al., 1985. Arch Biochem. Biophys. 240: 509-523). The later assay requires the presence of several detection enzymes.

Suitable enzymes for converting ATP into light include luciferases, e.g., insect luciferases. Luciferases produce light as an end-product of catalysis. The best known light-emitting enzyme is that of the firefly, Photinus pyralis (Coleoptera). The corresponding gene has been cloned and expressed in bacteria (see e.g., de Wet, et al., 1985. Proc. Natl. Acad. Sci. USA 80: 7870-7873) and plants (see e.g., Ow, et al, 1986. Science 234: 856-859), as well as in insect (see e.g., Jha, et al., 1990. FEBS Lett. 274: 24-26) and mammalian cells (see e.g., de Wet, et al, 1987. Mol. Cell. Biol. 7: 725-7373; Keller, et al., 1987. Proc. Natl. Acad. Sci. USA 82: 3264-3268). In addition, a number of luciferase genes from the Jamaican click beetle, Pyroplorus plagiophihalamus (Coleoptera), have recently been cloned and partially characterized (see e.g., Wood, et al., 1989. J. Biolumin. Chemilumin. 4: 289-301; Wood, et al., 1989. Science 244: 700-702). Distinct luciferases can sometimes produce light of different wavelengths, which may enable simultaneous monitoring of light emissions at different wavelengths. Accordingly, these aforementioned characteristics are unique, and add new dimensions with respect to the utilization of current reporter systems.

Firefly luciferase catalyzes bioluminescence in the presence of luciferin, adenosine 5′-triphosphate (ATP), magnesium ions, and oxygen, resulting in a quantum yield of 0.88 (see e.g., McElroy and Selinger, 1960. Arch. Biochem. Biophys. 88: 136-145). The firefly luciferase bioluminescent reaction can be utilized as an assay for the detection of ATP with a detection limit of approximately 1×10⁻¹³ M (see e.g., Leach, 1981. J. Appl. Biochem. 3: 473-517). In addition, the overall degree of sensitivity and convenience of the luciferase-mediated detection systems have created considerable interest in the development of firefly luciferase-based biosensors (see e.g., Green and Kricka, 1984. Talanta 31: 173-176; Blum, et al., 1989. J. Biolumin. Chemilumin. 4: 543-550).

The development of new reagents have made it possible to obtain stable light emission proportional to the concentrations of ATP (see e.g., Lundin, 1982. Applications of firefly luciferase In; Luminescent Assays (Raven Press, New York). With such stable light emission reagents, it is possible to make endpoint assays and to calibrate each individual assay by addition of a known amount of ATP. In addition, a stable light-emitting system also allows continuous monitoring of ATP-converting systems.

In a preferred embodiment, the ATP generating-ATP converting fusion protein is attached to an affinity tag. The term “affinity tag” is used herein to denote a peptide segment that can be attached to a polypeptide to provide for purification or detection of the polypeptide or provide sites for attachment of the polypeptide to a substrate. In principal, any peptide or protein for which an antibody or other specific binding agent is available can be used as an affinity tag. Affinity tags include a poly-histidine tract or a biotin carboxyl carrier protein (BCCP) domain, protein A (Nilsson et al., EMBO J. 4:1075, 1985; Nilsson et al., Methods Enzymol. 198:3, 1991), glutathione S transferase (Smith and Johnson, Gene 67:31, 1988), substance P, Flag.™. peptide (Hopp et al., Biotechnology 6:1204-1210, 1988; available from Eastman Kodak Co., New Haven, Conn.), streptavidin binding peptide, or other antigenic epitope or binding domain. See, in general Ford et al., Protein Expression and Purification 2: 95-107, 1991. DNAs encoding affinity tags are available from commercial suppliers (e.g., Pharmacia Biotech, Piscataway, N.J.).

As used herein, the term “poly-histidine tag,” when used in reference to a fusion protein refers to the presence of two to ten histidine residues at either the amino- or carboxy-terminus of a protein of interest. A poly-histidine tract of six to ten residues is preferred. The poly-histidine tract is also defined functionally as being a number of consecutive histidine residues added to the protein of interest which allows the affinity purification of the resulting fusion protein on a nickel-chelate or IDA column.

In some embodiments, the fusion protein has an orientation such that the sulfurylase polypeptide is N-terminal to the luciferase polypeptide. In other embodiments, the luciferase polypeptide is N-terminal to the sulfurylase polypeptide. As used herein, the term sulfurylase-luciferase fusion protein refers to either of these orientations. The terms “amino-terminal” (N-terminal) and “carboxyl-terminal” (C-terminal) are used herein to denote positions within polypeptides and proteins. Where the context allows, these terms are used with reference to a particular sequence or portion of a polypeptide or protein to denote proximity or relative position. For example, a certain sequence positioned carboxyl-terminal to a reference sequence within a protein is located proximal to the carboxyl terminus of the reference sequence, but is not necessarily at the carboxyl terminus of the complete protein.

The fusion protein of this invention can be produced by standard recombinant DNA techniques. For example, DNA fragments coding for the different polypeptide sequences are ligated together in-frame in accordance with conventional techniques, e.g., by employing blunt-ended or “sticky”-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). The two polypeptides of the fusion protein can also be joined by a linker, such as a unique restriction site, which is engineered with specific primers during the cloning procedure. In one embodiment, the sulfurylase and luciferase polypeptides are joined by a linker, for example an ala-ala-ala linker which is encoded by a Not1 restriction site.

In one embodiment, the invention includes a recombinant polynucleotide that comprises a coding sequence for a fusion protein having an ATP generating polypeptide sequence and an ATP converting polypeptide sequence. In a preferred embodiment, the recombinant polynucleotide encodes a sulfurylase-luciferase fusion protein. The term “recombinant DNA molecule” or “recombinant polynucleotide” as used herein refers to a DNA molecule which is comprised of segments of DNA joined together by means of molecular biological techniques. The term “recombinant protein” or “recombinant polypeptide” as used herein refers to a protein molecule which is expressed from a recombinant DNA molecule.

In one aspect, this invention discloses a sulfurylase-luciferase fusion protein with an N-terminal hexahistidine tag and a BCCP tag. The nucleic acid sequence of the disclosed N-terminal hexahistidine-BCCP luciferase-sulfurylase gene (His6-BCCP L-S) gene is shown below:

His6-BCCP L-S Nucleotide Sequence:

| | | |
|---|---|---|
| ATGCGGGGTTCTCATCATCATCATCATCATGGTATGGCTAGCATGGAAGCGCCAGCAGCA | 60 | (SEQ ID NO:3) |
| GCGGAAATCAGTGGTCACATCGTACGTTCCCCGATGGTTGGTACTTTCTACCGCACCCCA | 120 | |
| AGCCCGGACGCAAAAGCGTTCATCGAAGTGGGTCAGAAAGTCAACGTGGGCGATACCCTG | 180 | |
| TGCATCGTTGAAGCGATGAAAATGATGAACCAGATCGAAGCGGACAAATCCGGTACCGTG | 240 | |
| AAAGCAATTCTGGTCGAAAGTGGACAACCGGTAGAATTTGACGAGCCGCTGGTCGTCATC | 300 | |
| GAGGGATCCGAGCTCGAGATCCAAATGGAAGACGCCAAAAACATAAAGAAAGGCCCGGCG | 360 | |
| CCATTCTATCCTCTAGAGGATGGAACCGCTGGAGAGCAACTGCATAAGGCTATGAAGAGA | 420 | |
| TACGCCCTGGTTCCTGGAACAATTGCTTTTACAGATGCACATATCGAGGTGAACATCACG | 480 | |
| TACGCGGAATACTTCGAAATGTCCGTTCGGTTGGCAGAAGCTATGAAACGATATGGGCTG | 540 | |
| AATACAAATCACAGAATCGTCGTATGCAGTGAAAACTCTCTTCAATTCTTTATGCCGGTG | 600 | |
| TTGGGCGCGTTATTTATCGGAGTTGCAGTTGCGCCCGCGAACGACATTTATAATGAACGT | 660 | |
| GAATTGCTCAACAGTATGAACATTTCGCAGCCTACCGTAGTGTTTGTTTCCAAAAAGGGG | 720 | |
| TTGCAAAAAATTTTGAACGTGCAAAAAAAATTACCAATAATCCAGAAAATTATTATCATG | 780 | |
| GATTCTAAAACGGATTACCAGGGATTTCAGTCGATGTACACGTTCGTCACATCTCATCTA | 840 | |
| CCTCCCGGTTTTAATGAATACGATTTTGTACCAGAGTCCTTTGATCGTGACAAAACAATT | 900 | |
| GCACTGATAATGAATTCCTCTGGATCTACTGGGTTACCTAAGGGTGTGGCCCTTCCGCAT | 960 | |
| AGAACTGCCTGCGTCAGATTCTCGCATGCCAGAGATCCTATTTTTGGCAATCAAATCATT | 1020 | |
| CCGGATACTGCGATTTTAAGTGTTGTTCCATTCCATCACGGTTTTGGAATGTTTACTACA | 1080 | |
| CTCGGATATTTGATATGTGGATTTCGAGTCGTCTTAATGTATAGATTTGAAGAAGAGCTG | 1140 | |
| TTTTTACGATCCCTTCAGGATTACAAAATTCAAAGTGCGTTGCTAGTACCAACCCTATTT | 1200 | |
| TCATTCTTCGCCAAAAGCACTCTGATTGACAAATACGATTTATCTAATTTACACGAAATT | 1260 | |
| GCTTCTGGGGGCGCACCTCTTTCGAAAGAAGTCGGGGAAGCGGTTGCAAAACGCTTCCAT | 1320 | |
| CTTCCAGGGATACGACAAGGATATGGGCTCACTGAGACTACATCAGCTATTCTGATTACA | 1380 | |
| CCCGAGGGGGATGATAAACCGGGCGCGGTCGGTAAAGTTGTTCCATTTTTTGAAGCGAAG | 1440 | |
| GTTGTGGATCTGGATACCGGGAAAACGCTGGGCGTTAATCAGAGAGGCGAATTATGTGTC | 1500 | |
| AGAGGACCTATGATTATGTCCGGTTATGTAAACAATCCGGAAGCGACCAACGCCTTGATT | 1560 | |
| GACAAGGATGGATGGCTACATTCTGGAGACATAGCTTACTGGGACGAAGACGAACACTTC | 1620 | |
| TTCATAGTTGACCGCTTGAAGTCTTTAATTAAATACAAAGGATATCAGGTGGCCCCCGCT | 1680 | |
| GAATTGGAATCGATATTGTACAACACCCCAACATCTTTCGACGCGGGCGTGGCAGGTCTT | 1740 | |
| CCCGACGATGACGCCGGTGAACTTCCCGCCGCCGTTGTTGTTTTGGAGCACGGAAAGACG | 1800 | |
| ATGACGGAAAAAGAGATCGTGGATTACGTCGCCAGTCAAGTAACAACCGCGAAAAAGTTG | 1860 | |
| CGCGGAGGAGTTGTGTTTGTGGACGAAGTACCGAAAGGTCTTACCGGAAAACTCGACGCA | 1920 | |
| AGAAAAATCAGAGAGATCCTCATAAAGGCCAAGAAGGGCGGAAAGTCCAAATTGGCGGCC | 1980 | |
| GCTATGCCTGCTCCTCACGGTGGTATTCTACAAGACTTGATTGCTAGAGATGCGTTAAAG | 2040 | |
| AAGAATGAATTGTTATCTGAAGCGCAATCTTCGGACATTTTAGTATGGAACTTGACTCCT | 2100 | |
| AGACAACTATGTGATATTGAATTGATTCTAAATGGTGGGTTTTCTCCTCTGACTGGGTTT | 2160 | |
| TTGAACGAAAACGATTACTCCTCTGTTGTTACAGATTCGAGATTAGCAGACGGCACATTG | 2220 | |
| TGGACCATCCCTATTACATTAGATGTTGATGAAGCATTTGCTAACCAAATTAAACCAGAC | 2280 | |
| ACAAGAATTGCCCTTTTCCAAGATGATGAAATTCCTATTGCTATACTTACTGTCCAGGAT | 2340 | |
| GTTTACAAGCCAAACAAAACTATCGAAGCCGAAAAAGTCTTCAGAGGTGACCCAGAACAT | 2400 | |
| CCAGCCATTAGCTATTTATTTAACGTTGCCGGTGATTATTACGTCGGCGGTTCTTTAGAA | 2460 | |
| GCGATTCAATTACCTCAACATTATGACTATCCAGGTTTGCGTAAGACACCTGCCCAACTA | 2520 | |
| AGACTTGAATTCCAATCAAGACAATGGGACCGTGTCGTAGCTTTCCAAACTCGTAATCCA | 2580 | |
| ATGCATAGAGCCCACAGGGAGTTGACTGTGAGAGCCGCCAGAGAAGCTAATGCTAAGGTG | 2640 | |
| CTGATCCATCCAGTTGTTGGACTAACCAAACCAGGTGATATAGACCATCACACTCGTGTT | 2700 | |
| CGTGTCTACCAGGAAATTATTAAGCGTTATCCTAATGGTATTGCTTTCTTATCCCTGTTG | 2760 | |
| CCATTAGCAATGAGAATGAGTGGTGATAGAGAAGCCGTATGGCATGCTATTATTAGAAAG | 2820 | |
| AATTATGGTGCCTCCCACTTCATTGTTGGTAGAGACCATGCGGGCCCAGGTAAGAACTCC | 2880 | |
| AAGGGTGTTGATTTCTACGGTCCATACGATGCTCAAGAATTGGTCGAATCCTACAAGCAT | 2940 | |
| GAACTGGACATTGAAGTTGTTCCATTCAGAATGGTCACTTATTTGCCAGACGAAGACCGT | 3000 | |
| TATGCTCCAATTGATCAAATTGACACCACAAAGACGAGAACCTTGAACATTTCAGGTACA | 3060 | |
| GAGTTGAGACGCCGTTTAAGAGTTGGTGGTGAGATTCCTGAATGGTTCTCATATCCTGAA | 3120 | |
| GTGGTTAAAATCCTAAGAGAATCCAACCCACCAAGACCAAAACAAGGTTTTTCAATTGTT | 3180 | |
| TTAGGTAATTCATTAACCGTTTCTCGTGAGCAATTATCCATTGCTTTGTTGTCAACATTC | 3240 | |
| TTGCAATTCGGTGGTGGCAGGTATTACAAGATCTTTGAACACAATAATAAGACAGAGTTA | 3300 | |
| CTATCTTTGATTCAAGATTTCATTGGTTCTGGTAGTGGACTAATTATTCCAAATCAATGG | 3360 | |
| GAAGATGACAAGGACTCTGTTGTTGGCAAGCAAAACGTTTACTTATTAGATACCTCAAGC | 3420 | |
| TCAGCCGATATTCAGCTAGAGTCAGCGGATGAACCTATTTCACATATTGTACAAAAAGTT | 3480 | |
| GTCCTATTCTTGGAAGACAATGGCTTTTTTGTATTTTAA | 3519 | |

The amino acid sequence of the disclosed His6-BCCP L-S polypeptide is presented using the three letter amino acid code (SEQ ID NO:4).

His6-BCCP L-S Amino Acid Sequence

| Met Arg Gly Ser His His His His His His Gly Met Ala Ser Met Glu | (SEQ ID NO:4) |
|---|---|
| 1 5 10 15 | |
| Ala Pro Ala Ala Ala Glu Ile Ser Gly His Ile Val Arg Ser Pro Met | |
| 20 25 30 | |
| Val Gly Thr Phe Tyr Arg Thr Pro Ser Pro Asp Ala Lys Ala Phe Ile | |
| 35 40 45 | |
| Glu Val Gly Gln Lys Val Asn Val Gly Asp Thr Leu Cys Ile Val Glu | |
| 50 55 60 | |
| Ala Met Lys Met Met Asn Gln Ile Glu Ala Asp Lys Ser Gly Thr Val | |
| 65 70 75 80 | |
| Lys Ala Ile Leu Val Glu Ser Gly Gln Pro Val Glu Phe Asp Glu Pro | |
| 85 90 95 | |
| Leu Val Val Ile Glu Gly Ser Glu Leu Glu Ile Gln Met Glu Asp Ala | |
| 100 105 110 | |
| Lys Asn Ile Lys Lys Gly Pro Ala Pro Phe Tyr Pro Leu Glu Asp Gly | |
| 115 120 125 | |
| Thr Ala Gly Glu Gln Leu His Lys Ala Met Lys Arg Tyr Ala Leu Val | |
| 130 135 140 | |
| Pro Gly Thr Ile Ala Phe Thr Asp Ala His Ile Glu Val Asn Ile Thr | |
| 145 150 155 160 | |
| Tyr Ala Glu Tyr Phe Glu Met Ser Val Arg Leu Ala Glu Ala Met Lys | |
| 165 170 175 | |
| Arg Tyr Gly Leu Asn Thr Asn His Arg Ile Val Val Cys Ser Glu Asn | |
| 180 185 190 | |
| Ser Leu Gln Phe Phe Met Pro Val Leu Gly Ala Leu Phe Ile Gly Val | |
| 195 200 205 | |
| Ala Val Ala Pro Ala Asn Asp Ile Tyr Asn Glu Arg Glu Leu Leu Asn | |
| 210 215 220 | |
| Ser Met Asn Ile Ser Gln Pro Thr Val Val Phe Val Ser Lys Lys Gly | |
| 225 230 235 240 | |
| Leu Gln Lys Ile Leu Asn Val Gln Lys Lys Leu Pro Ile Ile Gln Lys | |
| 245 250 255 | |
| Ile Ile Ile Met Asp Ser Lys Thr Asp Tyr Gln Gly Phe Gln Ser Met | |
| 260 265 270 | |
| Tyr Thr Phe Val Thr Ser His Leu Pro Pro Gly Phe Asn Glu Tyr Asp | |
| 275 280 285 | |
| Phe Val Pro Glu Ser Phe Asp Arg Asp Lys Thr Ile Ala Leu Ile Met | |
| 290 295 300 | |
| Asn Ser Ser Gly Ser Thr Gly Leu Pro Lys Gly Val Ala Leu Pro His | |
| 305 310 315 320 | |
| Arg Thr Ala Cys Val Arg Phe Ser His Ala Arg Asp Pro Ile Phe Gly | |
| 325 330 335 | |
| Asn Gln Ile Ile Pro Asp Thr Ala Ile Leu Ser Val Val Pro Phe His | |
| 340 345 350 | |
| His Gly Phe Gly Met Phe Thr Thr Leu Gly Tyr Leu Ile Cys Gly Phe | |
| 355 360 365 | |
| Arg Val Val Leu Met Tyr Arg Phe Glu Glu Glu Leu Phe Leu Arg Ser | |
| 370 375 380 | |
| Leu Gln Asp Tyr Lys Ile Gln Ser Ala Leu Leu Val Pro Thr Leu Phe | |
| 385 390 395 400 | |
| Ser Phe Phe Ala Lys Ser Thr Leu Ile Asp Lys Tyr Asp Leu Ser Asn | |
| 405 410 415 | |
| Leu His Glu Ile Ala Ser Gly Gly Ala Pro Leu Ser Lys Glu Val Gly | |
| 420 425 430 | |
| Glu Ala Val Ala Lys Arg Phe His Leu Pro Gly Ile Arg Gln Gly Tyr | |
| 435 440 445 | |
| Gly Leu Thr Glu Thr Thr Ser Ala Ile Leu Ile Thr Pro Glu Gly Asp | |
| 450 455 460 | |
| Asp Lys Pro Gly Ala Val Gly Lys Val Val Pro Phe Phe Glu Ala Lys | |
| 465 470 475 480 | |
| Val Val Asp Leu Asp Thr Gly Lys Thr Leu Gly Val Asn Gln Arg Gly | |
| 485 490 495 | |
| Glu Leu Cys Val Arg Gly Pro Met Ile Met Ser Gly Tyr Val Asn Asn | |
| 500 505 510 | |
| Pro Glu Ala Thr Asn Ala Leu Ile Asp Lys Asp Gly Tyr Leu His Ser | |
| 515 520 525 | |
| Gly Asp Ile Ala Tyr Trp Asp Glu Asp Glu His Phe Phe Ile Val Asp | |
| 530 535 540 | |
| Arg Leu Lys Ser Leu Ile Lys Tyr Lys Gly Tyr Gln Val Ala Pro Ala | |
| 545 550 555 560 | |
| Glu Leu Glu Ser Ile Leu Leu Gln His Pro Asn Ile Phe Asp Ala Gly | |
| 565 570 575 | |
| Val Ala Gly Leu Pro Asp Asp Asp Ala Gly Glu Leu Pro Ala Ala Val | |
| 580 585 590 | |
| Val Val Leu Glu His Gly Lys Thr Met Thr Glu Lys Glu Ile Val Asp | |
| 595 600 605 | |
| Tyr Val Ala Ser Gln Val Thr Thr Ala Lys Lys Leu Arg Gly Gly Val | |
| 610 615 620 | |
| Val Phe Val Asp Glu Val Pro Lys Gly Leu Thr Gly Lys Leu Asp Ala | |
| 625 630 635 640 | |
| Arg Lys Ile Arg Glu Ile Leu Ile Lys Ala Lys Lys Gly Gly Lys Ser | |
| 645 650 655 | |
| Lys Leu Ala Ala Ala Met Pro Ala Pro His Gly Gly Ile Leu Gln Asp | |
| 660 665 670 | |
| Leu Ile Ala Arg Asp Ala Leu Lys Lys Asn Glu Leu Leu Ser Glu Ala | |
| 675 680 685 | |
| Gln Ser Ser Asp Ile Leu Val Trp Asn Leu Thr Pro Arg Gln Leu Cys | |
| 690 695 700 | |
| Asp Ile Glu Leu Ile Leu Asn Gly Gly Phe Ser Pro Leu Thr Gly Phe | |
| 705 710 715 | |
| Leu Asn Glu Asn Asp Tyr Ser Ser Val Val Thr Asp Ser Arg Leu Ala | |
| 720 725 730 735 | |
| Asp Gly Thr Leu Trp Thr Ile Pro Ile Thr Leu Asp Val Asp Glu Ala | |
| 740 745 750 | |
| Phe Ala Asn Gln Ile Lys Pro Asp Thr Arg Ile Ala Leu Phe Gln Asp | |
| 755 760 765 | |
| Asp Glu Ile Pro Ile Ala Ile Leu Thr Val Gln Asp Val Tyr Lys Pro | |
| 770 775 780 | |
| Asn Lys Thr Ile Glu Ala Glu Lys Val Phe Arg Gly Asp Pro Glu His | |
| 785 790 795 | |
| Pro Ala Ile Ser Tyr Leu Phe Asn Val Ala Gly Asp Tyr Tyr Val Gly | |
| 800 805 810 815 | |
| Gly Ser Leu Glu Ala Ile Gln Leu Pro Gln His Tyr Asp Tyr Pro Gly | |
| 820 825 830 | |
| Leu Arg Lys Thr Pro Ala Gln Leu Arg Leu Glu Phe Gln Ser Arg Gln | |
| 835 840 845 | |
| Trp Asp Arg Val Val Ala Phe Gln Thr Arg Asn Pro Met His Arg Ala | |
| 850 855 860 | |
| His Arg Glu Leu Thr Val Arg Ala Ala Arg Glu Ala Asn Ala Lys Val | |
| 865 870 875 | |
| Leu Ile His Pro Val Val Gly Leu Thr Lys Pro Gly Asp Ile Asp His | |
| 880 885 890 895 | |
| His Thr Arg Val Arg Val Tyr Gln Glu Ile Ile Lys Arg Tyr Pro Asn | |
| 900 905 910 | |
| Gly Ile Ala Phe Leu Ser Leu Leu Pro Leu Ala Met Arg Met Ser Gly | |
| 915 920 925 | |
| Asp Arg Glu Ala Val Trp His Ala Ile Ile Arg Lys Asn Tyr Gly Ala | |
| 930 935 940 | |
| Ser His Phe Ile Val Gly Arg Asp His Ala Gly Pro Gly Lys Asn Ser | |
| 945 950 955 | |
| Lys Gly Val Asp Phe Tyr Gly Pro Tyr Asp Ala Gln Glu Leu Val Glu | |
| 960 965 970 975 | |
| Ser Tyr Lys His Glu Leu Asp Ile Glu Val Val Pro Phe Arg Met Val | |
| 980 985 990 | |
| Thr Tyr Leu Pro Asp Glu Asp Arg Tyr Ala Pro Ile Asp Gln Ile Asp | |
| 995 1000 1005 | |
| Thr Thr Lys Thr Arg Thr Leu Asn Ile Ser Gly Thr Glu Leu Arg Arg | |
| 1010 1015 1020 | |
| Arg Leu Arg Val Gly Gly Glu Ile Pro Glu Trp Phe Ser Tyr Pro Glu | |
| 1025 1030 1035 | |
| Val Val Lys Ile Leu Arg Glu Ser Asn Pro Pro Arg Pro Lys Gln Gly | |
| 1040 1045 1050 1055 | |
| Phe Ser Ile Val Leu Gly Asn Ser Leu Thr Val Ser Arg Glu Gln Leu | |
| 1060 1065 1070 | |
| Ser Ile Ala Leu Leu Ser Thr Phe Leu Gln Phe Gly Gly Gly Arg Tyr | |
| 1075 1080 1085 | |
| Tyr Lys Ile Phe Glu His Asn Asn Lys Thr Glu Leu Leu Ser Leu Ile | |
| 1090 1095 1100 | |
| Gln Asp Phe Ile Gly Ser Gly Ser Gly Leu Ile Ile Pro Asn Gln Trp | |
| 1105 1110 1115 | |
| Glu Asp Asp Lys Asp Ser Val Val Gly Lys Gln Asn Val Tyr Leu Leu | |
| 1120 1125 1130 1135 | |
| Asp Thr Ser Ser Ser Ala Asp Ile Gln Leu Glu Ser Ala Asp Glu Pro | |
| 1140 1145 1150 | |
| Ile Ser His Ile Val Gln Lys Val Val Leu Phe Leu Glu Asp Asn Gly | |
| 1155 1160 1165 | |
| Phe Phe Val Phe | |
| 1170 | |

Accordingly, in one aspect, the invention provides for a fusion protein comprising a thermostable sulfurylase joined to at least one affinity tag. The nucleic acid sequence of the disclosed N-terminal hexahistidine-BCCP Bst ATP Sulfurylase (His6-BCCP Bst Sulfurylase) gene is shown below:

His6-BCCP Bst Sulfurylase Nucleotide Sequence

| | | |
|---|---|---|
| ATGCGGGGTTCTCATCATCATCATCATCATGGTATGGCTAGCATGGAAGCGCCAGCAGCA | 60 | (SEQ ID NO:5) |
| GCGGAAATCAGTGGTCACATCGTACGTTCCCCGATGGTTGGTACTTTCTACCGCACCCCA | 120 | |
| AGCCCGGACGCAAAAGCGTTCATCGAAGTGGGTCAGAAAGTCAACGTGGGCGATACCCTG | 180 | |
| TGCATCGTTGAAGCCATGAAAATGATGAACCAGATCGAAGCGGACAAATCCGGTACCGTG | 240 | |
| AAAGCAATTCTGGTCGAAAGTGGACAACCGGTAGAATTTGACGAGCCGCTGGTCGTCATC | 300 | |
| GAGGGATCCGAGCTCGAGATCTGCAGCATGAGCGTAAGCATCCCGCATGGCGGCACATTG | 360 | |
| ATCAACCGTTGGAATCCGGATTACCCAATCGATGAAGCAACGAAAACGATCGAGCTGTCC | 420 | |
| AAAGCCGAACTAAGCGACCTTGAGCTGATCGGCACAGGCGCCTACAGCCCGCCCACCGGG | 480 | |
| TTTTTAACGAAAGCCGATTACGATGCGGTCGTAGAAACGATGCGCCTCGCTGATGGCACT | 540 | |
| GTCTGGAGCATTCCGATCACGCTGGCGGTGACGGAAGAAAAAGCGAGTGAACTCACTGTC | 600 | |
| GGCGACAAAGCGAAACTCGTTTATGGCGGCGACGTCTACGGCGTCATTGAAATCGCCGAT | 660 | |
| ATTTACCGCCCGGATAAAACGAAAGAAGCCAAGCTCGTCTATAAAACCGATGAACTCGCT | 720 | |
| CACCCGGGCGTGCGCAAGCTGTTTGAAAAACCAGATGTGTACGTCGGCGGAGCGGTTACG | 780 | |
| CTCGTCAAACGGACCGACAAAGGCCAGTTTGCTCCGTTTTATTTCGATCCGGCCGAAACG | 840 | |
| CGGAAACGATTTGCCGAACTCGGCTGGAATACCGTCGTCGGCTTCCAAACACGCAACCCG | 900 | |
| GTTCACCGCGCCCATGAATACATTCAAAAATGCGCGCTTGAAATCGTGGACGGCTTGTTT | 960 | |
| TTAAACCCGCTCGTCGGCGAAACGAAAGCGGACGATATTCCGGCCGACATCCGGATGGAA | 1020 | |
| AGCTATCAAGTGCTGCTGGAAAACTATTATCCGAAAGACCGCGTTTTCTTGGGCGTCTTC | 1080 | |
| CAAGCTGCGATGCGCTATGCCGGTCCGCGCGAAGCGATTTTCCATGCCATGGTGCGGAAA | 1140 | |
| AACTTCGGCTGCACGCACTTCATCGTCGGCCGCGACCATGCGGGCGTCGGCAACTATTAC | 1200 | |
| GGCACGTATGATGCGCAAAAAATCTTCTCGAACTTTACAGCCGAAGAGCTTGGCATTACA | 1260 | |
| CCGCTCTTTTTCGAACACAGCTTTTATTGCACGAAATGCGAAGGCATGGCATCGACGAAA | 1320 | |
| ACATGCCCGCACGACGCACAATATCACGTTGTCCTTTCTGGCACGAAAGTCCGTGAAATG | 1380 | |
| TTGCGTAACGGCCAAGTGCCGCCGAGCACATTCAGCCGTCCGGAAGTGGCCGCCGTTTTG | 1440 | |
| ATCAAAGGGCTGCAAGAACGCGAAACGGTCGCCCCGTCAGCGCGCTAA | 1488 | |

The amino acid sequence of the His6-BCCP Bst Sulfurylase polypeptide is presented using the three letter amino acid code in Table 6 (SEQ ID NO:6).

His6-BCCP Bst Sulfurylase Amino Acid Sequence

| Met Arg Gly Ser His His His His His His Gly Met Ala Ser Met Glu | (SEQ ID NO:6) |
|---|---|
| 1 5 10 15 | |
| Ala Pro Ala Ala Ala Glu Ile Ser Gly His Ile Val Arg Ser Pro Met | |
| 20 25 30 | |
| Val Gly Thr Phe Tyr Arg Thr Pro Ser Pro Asp Ala Lys Ala Phe Ile | |
| 35 40 45 | |
| Glu Val Gly Gln Lys Val Asn Val Gly Asp Thr Leu Cys Ile Val Glu | |
| 50 55 60 | |
| Ala Met Lys Met Met Asn Gln Ile Glu Ala Asp Lys Ser Gly Thr Val | |
| 65 70 75 80 | |
| Lys Ala Ile Leu Val Glu Ser Gly Gln Pro Val Glu Phe Asp Glu Pro | |
| 85 90 95 | |
| Leu Val Val Ile Glu Gly Ser Glu Leu Glu Ile Cys Ser Met Ser Val | |
| 100 105 110 | |
| Ser Ile Pro His Gly Gly Thr Leu Ile Asn Arg Trp Asn Pro Asp Tyr | |
| 115 120 125 | |
| Pro Ile Asp Glu Ala Thr Lys Thr Ile Glu Leu Ser Lys Ala Glu Leu | |
| 130 135 140 | |
| Ser Asp Leu Glu Leu Ile Gly Thr Gly Ala Tyr Ser Pro Leu Thr Gly | |
| 145 150 155 160 | |
| Phe Leu Thr Lys Ala Asp Tyr Asp Ala Val Val Glu Thr Met Arg Leu | |
| 165 170 175 | |
| Ala Asp Gly Thr Val Trp Ser Ile Pro Ile Thr Leu Ala Val Thr Gln | |
| 180 185 190 | |
| Glu Lys Ala Ser Gln Leu Thr Val Gly Asp Lys Ala Lys Leu Val Tyr | |
| 195 200 205 | |
| Gly Gly Asp Val Tyr Gly Val Ile Glu Ile Ala Asp Ile Tyr Arg Pro | |
| 210 215 220 | |
| Asp Lys Thr Lys Glu Ala Lys Leu Val Tyr Lys Thr Asp Gln Leu Ala | |
| 225 230 235 240 | |
| His Pro Gly Val Arg Lys Leu Phe Gln Lys Pro Asp Val Tyr Val Gly | |
| 245 250 255 | |
| Gly Ala Val Thr Leu Val Lys Arg Thr Asp Lys Gly Gln Phe Ala Pro | |
| 260 265 270 | |
| Phe Tyr Phe Asp Pro Ala Glu Thr Arg Lys Arg Phe Ala Glu Leu Gly | |
| 275 280 285 | |
| Trp Asn Thr Val Val Gly Phe Gln Thr Arg Asn Pro Val His Arg Ala | |
| 290 295 300 | |
| His Glu Tyr Ile Gln Lys Cys Ala Leu Glu Ile Val Asp Gly Leu Phe | |
| 305 310 315 320 | |
| Leu Asn Pro Leu Val Gly Glu Thr Lys Ala Asp Asp Ile Pro Ala Asp | |
| 325 330 335 | |
| Ile Arg Met Glu Ser Tyr Gln Val Leu Leu Glu Asn Tyr Tyr Pro Lys | |
| 340 345 350 | |
| Asp Arg Val Phe Leu Gly Val Phe Gln Ala Ala Met Arg Tyr Ala Gly | |
| 355 360 365 | |
| Pro Arg Glu Ala Ile Phe His Ala Met Val Arg Lys Asn Phe Gly Cys | |
| 370 375 380 | |
| Thr His Phe Ile Val Gly Arg Asp His Ala Gly Val Gly Asn Tyr Tyr | |
| 385 390 395 400 | |
| Gly Thr Tyr Asp Ala Gln Lys Ile Phe Ser Asn Phe Thr Ala Glu Glu | |
| 405 410 415 | |
| Leu Gly Ile Thr Pro Leu Phe Phe Glu His Ser Phe Tyr Cys Thr Lys | |
| 420 425 430 | |
| Cys Glu Gly Met Ala Ser Thr Lys Thr Cys Pro His Asp Ala Gln Tyr | |
| 435 440 445 | |
| His Val Val Leu Ser Gly Thr Lys Val Arg Glu Met Leu Arg Asn Gly | |
| 450 455 460 | |
| Gln Val Pro Pro Ser Thr Phe Ser Arg Pro Glu Val Ala Ala Val Leu | |
| 465 470 475 480 | |
| Ile Lys Gly Leu Gln Glu Arg Glu Thr Val Ala Pro Ser Ala Arg | |
| 485 490 495 | |

Another aspect of the invention pertains to vectors, preferably expression vectors, containing a nucleic acid encoding an ATP generating polypeptide and an ATP converting polypeptide, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce a fusion protein.

The recombinant expression vectors of the invention can be designed for expression of the fusion protein in prokaryotic or eukaryotic cells. For example, a sulfurylase-luciferase fusion protein can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein.

In another embodiment, the ATP generating-ATP converting fusion protein expression vector is a yeast expression vector. Examples of vectors for expression in yeast S. cerivisae include pYepSec1 (Baldari, et al., (1987) EMBO J 6:229-234), pMFa (Kouan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the fusion protein can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The invention also includes a kit comprising a sulfurylase-luciferase fusion protein expression vector.

A host cell can be any prokaryotic or eukaryotic cell. For example, the sulfurylase-luciferase fusion protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding ORFX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the fusion protein. Accordingly, the invention further provides methods for producing the fusion protein using the host cells of the invention. In one embodiment, the method comprises culturing the host cell of invention (into which a recombinant expression vector encoding the fusion protein has been introduced) in a suitable medium such that the fusion protein is produced. In another embodiment, the method further comprises isolating the fusion protein from the medium or the host cell.

The invention also includes a fusion protein bound to a mobile support. In a preferred embodiment, the fusion gene is a sulfurylase-luciferase fusion gene. In another embodiment, the mobile support is bound to strepavidin. The mobile support could be a bead or optical fiber. In a preferred embodiment, the bead is a nickel-agarose bead or a MPG-Streptavidin bead. In one embodiment, the sulfurylase-luciferase fusion protein is bound to the beads in a 1:3 ratio of protein to bead. It can be attached to the solid support via a covalent or non-covalent interaction. In general, any linkage recognized in the art can be used. Examples of such linkages common in the art include any suitable metal (e.g., Co²⁺, Ni²⁺)-hexahistidine complex, a biotin binding protein, e.g., NEUTRAVIDIN™ modified avidin (Pierce Chemicals, Rockford, Ill.), streptavidin/biotin, avidin/biotin, glutathione S-transferase (GST)/glutathione, monoclonal antibody/antigen, and maltose binding protein/maltose, and pluronic coupling technologies. Samples containing the appropriate tag are incubated with the sensitized substrate so that zero, one, or multiple molecules attach at each sensitized site.

Acetyl-CoA carboxylase (ACCase) catalyzes the first committed step in de novo fatty acid biosynthesis. It belongs to a group of carboxylases that use biotin as cofactor and bicarbonate as a source of the carboxyl group. There are two types of ACCase: prokaryotic ACCase (e.g., E. coli, P. aeruginosa, Anabaena, Synechococcus and probably pea chloroplast) in which the three functional domains: biotin carboxylase (BC), biotin carboxyl carrier protein (BCCP) and carboxyltransferase (CT) are located on separable subunits and eukaryotic ACCase (e.g., rat, chicken, yeast, diatom and wheat) in which all the domains are located on one large polypeptide. It is known that a BCCP as a subunit of acetyl CoA carboxylase from E. coli is biotinated at the Lys residue at the 122-position by the action of biotin holoenzyme synthetase in E. coli (Journal of Biological Chemistry, 263, 6461 (1988)). In a preferred embodiment of this invention, the fusion protein is bound to a BCCP domain which is then utilized for binding avidins; therefore, it can bind to a streptavidin mobile support. One biotin-(strept-)avidin-based anchoring method uses a thin layer of a photoactivatable biotin analog dried onto a solid surface. (Hengsakul and Cass, 1996. Bioconjugate Chem. 7: 249-254). The biotin analog is then exposed to white light through a mask, so as to create defined areas of activated biotin. Avidin (or streptavidin) is then added and allowed to bind to the activated biotin. The avidin possesses free biotin binding sites which can be utilized to “anchor” the biotinylated proteins through a biotin-(strept-)avidin linkage.

Alternatively, the fusion protein can be attached to the solid support with a biotin derivative possessing a photo-removable protecting group. This moiety is covalently bound to bovine serum albumin (BSA), which is attached to the solid support, e.g., a glass surface. See Pirrung and Huang, 1996. Bioconjugate Chem. 7: 317-321. A mask is then used to create activated biotin within the defined irradiated areas. Avidin may then be localized to the irradiated area, with a biotinylated sulfurylase-luciferase fusion protein subsequently attached through a BSA-biotin-avidin-biotin link.

Another method of attachment is with the use of a pluronics based attachment. Pluronics attach to hydrophobic surfaces by virtue of the reaction between the hydrophobic surface and the polypropylene oxide. The remaining polyethylene oxide groups extend off the surface, thereby creating a hydrophilic environment. Nitrilotriacetic acid (NTA) can be conjugated to the terminal ends of the polyethylene oxide chains to allow for hexahistidine tagged proteins to be attached.

This invention provides methods of sequencing which utilize and ATP generating polypeptide-ATP converting polypeptide fusion protein for detection. In a preferred embodiment, the nucleotide sequence of the sequencing product is determined by measuring inorganic pyrophosphate (PPi) liberated from a nucleotide triphosphate (dNTP) as the dNMP is incorporated into an extended sequence primer. This method of sequencing is termed Pyrosequencing™ technology (PyroSequencing AB, Stockholm, Sweden). It can be performed in solution (liquid phase) or as a solid phase technique. Various sequencing methods, including PPi sequencing methods, are described in, e.g., WO9813523A1, Ronaghi, et al., 1996. Anal. Biochem. 242: 84-89, and Ronaghi, et al, 1998. Science 281: 363-365 (1998), U.S. Pat. No. 6,274,320 and the patent application U.S. Ser. No. 10/104,280 which was filed on Mar. 21, 2001 (21465-501CIP3). These disclosures of sequencing are incorporated herein in their entirety, by reference.

Pyrophosphate released under these conditions can be detected enzymatically (e.g., by the generation of light in the luciferase-luciferin reaction). Such methods enable a nucleotide to be identified in a given target position, and the DNA to be sequenced simply and rapidly while avoiding the need for electrophoresis and the use of potentially dangerous radiolabels.

The invention also provides a method for sequencing nucleic acids which generally comprises (a) providing one or more nucleic acid anchor primers and a plurality of single-stranded circular nucleic acid templates disposed within a plurality of reaction chambers or cavities; (b) annealing an effective amount of the nucleic acid anchor primer to at least one of the single-stranded circular templates to yield a primed anchor primer-circular template complex; (c) combining the primed anchor primer-circular template complex with a polymerase to form an extended anchor primer covalently linked to multiple copies of a nucleic acid complementary to the circular nucleic acid template; (d) annealing an effective amount of a sequencing primer to one or more copies of said covalently linked complementary nucleic acid; (e) extending the sequencing primer with a polymerase and a predetermined nucleotide triphosphate to yield a sequencing product and, if the predetermined nucleotide triphosphate is incorporated onto the 3′ end of said sequencing primer, a sequencing reaction byproduct; and (f) identifying the PPi sequencing reaction byproduct with the use of an ATP generating polypeptide-ATP converting polypeptide fusion protein, thereby determining the sequence of the nucleic acid. In one embodiment, a dATP or ddATP analogue is used in place of deoxy- or dideoxy adenosine triphosphate. This analogue is capable of acting as a substrate for a polymerase but incapable of acting as a substrate for a PPi-detection enzyme. This method can be carried out in separate parallel common reactions in an aqueous environment.

In another aspect, the invention includes a method of determining the base sequence of a plurality of nucleotides on an array, which generally comprises (a) providing a plurality of sample DNAs, each disposed within a plurality of cavities on a planar surface; (b) adding an activated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates; (c) utilizing an ATP generating polypeptide-ATP converting polypeptide fusion protein to detect whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor; and (d) sequentially repeating steps (b) and (c), wherein each sequential repetition adds and, detects the incorporation of one type of activated nucleoside 5′-triphosphate precursor of known nitrogenous base composition; and (e) determining the base sequence of the unpaired nucleotide residues of the template in each reaction chamber from the sequence of incorporation of said nucleoside precursors.

The anchor primers of the invention generally comprise a stalk region and at least one adaptor region. In a preferred embodiment the anchor primer contains at least two contiguous adapter regions. The stalk region is present at the 5′ end of the anchor primer and includes a region of nucleotides for attaching the anchor primer to the solid substrate.

The adaptor region(s) comprise nucleotide sequences that hybridize to a complementary sequence present in one or more members of a population of nucleic acid sequences. In some embodiments, the anchor primer includes two adjoining adaptor regions, which hybridize to complementary regions ligated to separate ends of a target nucleic acid sequence. In additional embodiments, the adapter regions in the anchor primers are complementary to non-contiguous regions of sequence present in a second nucleic acid sequence. Each adapter region, for example, can be homologous to each terminus of a fragment produced by digestion with one or more restriction endonucleases. The fragment can include, e.g., a sequence known or suspected to contain a sequence polymorphism. Additionally, the anchor primer may contain two adapter regions that are homologous to a gapped region of a target nucleic acid sequence, i.e., one that is non-contiguous because of a deletion of one or more nucleotides. When adapter regions having these sequences are used, an aligning oligonucleotide corresponding to the gapped sequence may be annealed to the anchor primer along with a population of template nucleic acid molecules.

The anchor primer may optionally contain additional elements such as one or more restriction enzyme recognition sites, RNA polymerase binding sites, e.g., a T7 promoter site, or sequences present in identified DNA sequences, e.g., sequences present in known genes. The adapter region(s) may also include sequences known to flank sequence polymorphisms. Sequence polymorphisms include nucleotide substitutions, insertions, deletions, or other rearrangements which result in a sequence difference between two otherwise identical nucleic acid sequences. An example of a sequence polymorphism is a single nucleotide polymorphism (SNP).

In general, any nucleic acid capable of base-pairing can be used as an anchor primer. In some embodiments, the anchor primer is an oligonucleotide. As utilized herein the term oligonucleotide includes linear oligomers of natural or modified monomers or linkages, e.g., deoxyribonucleosides, ribonucleosides, anomeric forms thereof, peptide nucleic acids (PNAs), and the like, that are capable of specifically binding to a target polynucleotide by way of a regular pattern of monomer-to-monomer interactions. These types of interactions can include, e.g., Watson-Crick type of base-pairing, base stacking, Hoogsteen or reverse-Hoogsteen types of base-pairing, or the like. Generally, the monomers are linked by phosphodiester bonds, or analogs thereof, to form oligonucleotides ranging in size from, e.g., 3-200, 8-150, 10-100, 20-80, or 25-50 monomeric units. Whenever an oligonucleotide is represented by a sequence of letters, it is understood that the nucleotides are oriented in the 5′→3′ direction, from left-to-right, and that the letter “A” donates deoxyadenosine, the letter “T” denotes thymidine, the letter “C” denotes deoxycytosine, and the letter “G” denotes deoxyguanosine, unless otherwise noted herein. The oligonucleotides of the present invention can include non-natural nucleotide analogs. However, where, for example, processing by enzymes is required, or the like, oligonucleotides comprising naturally occurring nucleotides are generally required for maintenance of biological function.

Anchor primers are linked to the solid substrate at the sensitized sites. They can be linked by the same method of linkage as described for the fusion protein to the solid support. A region of a solid substrate containing a linked primer is referred to herein as an anchor pad. Thus, by specifying the sensitized states on the solid support, it is possible to form an array or matrix of anchor pads. The anchor pads can be, e.g., small diameter spots etched at evenly spaced intervals on the solid support. The anchor pads can be located at the bottoms of the cavitations or wells if the substrate has been cavitated, etched, or otherwise micromachined as discussed above.

In one embodiment, the anchor primer is linked to a particle. The anchor primer can be linked to the particle prior to formation of the extended anchor primer or after formation of the extended anchor primer.

Each sensitized site on a solid support is potentially capable of attaching multiple anchor primers. Thus, each anchor pad may include one or more anchor primers. It is preferable to maximize the number of pads that have only a single productive reaction center (e.g., the number of pads that, after the extension reaction, have only a single sequence extended from the anchor primer). This can be accomplished by techniques which include, but are not limited to: (i) varying the dilution of biotinylated anchor primers that are washed over the surface; (ii) varying the incubation time that the biotinylated primers are in contact with the avidin surface; (iii) varying the concentration of open- or closed-circular template so that, on average, only one primer on each pad is extended to generate the sequencing template; or (iv) reducing the size of the anchor pad to approach single-molecule dimensions (<1 μm) such that binding of one anchor inhibits or blocks the binding of another anchor (e.g. by photoactivation of a small spot); or (v) reducing the size of the anchor pad such that binding of one circular template inhibits or blocks the binding of a second circular template.

In some embodiments, each individual pad contains just one linked anchor primer. Pads having only one anchor primer can be made by performing limiting dilutions of a selected anchor primer on to the solid support such that, on average, only one anchor primer is deposited on each pad. The concentration of anchor primer to be applied to a pad can be calculated utilizing, for example, a Poisson distribution model.

In order to maximize the number of reaction pads that contain a single anchor primer, a series of dilution experiments are performed in which a range of anchor primer concentrations or circular template concentrations are varied. For highly dilute concentrations of primers, primers and circular templates binding to the same pad will be independent of each other, and a Poisson distribution will characterize the number of anchor primers extended on any one pad. Although there will be variability in the number of primers that are actually extended, a maximum of 37% of the pads will have a single extended anchor primer (the number of pads with a single anchor oligonucleotide).

In other embodiments multiple anchor primers are attached to any one individual pad in an array. Limiting dilutions of a plurality of circular nucleic acid templates (described in more detail below) may be hybridized to the anchor primers so immobilized such that, on average, only one primer on each pad is hybridized to a nucleic acid template. Library concentrations to be used may be calculated utilizing, for example, limiting dilutions and a Poisson distribution model.

The nucleic acid templates that can be sequenced according to the invention, e.g., a nucleic acid library, in general can include open circular or closed circular nucleic acid molecules. A “closed circle” is a covalently closed circular nucleic acid molecule, e.g., a circular DNA or RNA molecule. An “open circle” is a linear single-stranded nucleic acid molecule having a 5′ phosphate group and a 3′ hydroxyl group. In one embodiment, the single stranded nucleic acid contains at least 100 copies of nucleic acid sequence, each copy covalently linked end to end. In some embodiments, the open circle is formed in situ from a linear double-stranded nucleic acid molecule. The ends of a given open circle nucleic acid molecule can be ligated by DNA ligase. Sequences at the 5′ and 3′ ends of the open circle molecule are complementary to two regions of adjacent nucleotides in a second nucleic acid molecule, e.g., an adapter region of an anchor primer, or to two regions that are nearly adjoining in a second DNA molecule. Thus, the ends of the open-circle molecule can be ligated using DNA ligase, or extended by DNA polymerase in a gap-filling reaction. Open circles are described in detail in Lizardi, U.S. Pat. No. 5,854,033. An open circle can be converted to a closed circle in the presence of a DNA ligase (for DNA) or RNA ligase following, e.g., annealing of the open circle to an anchor primer.

If desired, nucleic acid templates can be provided as padlock probes. Padlock probes are linear oligonucleotides that include target-complementary sequences located at each end, and which are separated by a linker sequence. The linkers can be ligated to ends of members of a library of nucleic acid sequences that have been, e.g., physically sheared or digested with restriction endonucleases. Upon hybridization to a target-sequence, the 5′- and 3′-terminal regions of these linear oligonucleotides are brought in juxtaposition. This juxtaposition allows the two probe segments (if properly hybridized) to be covalently-bound by enzymatic ligation (e.g., with T4 DNA ligase), thus converting the probes to circularly-closed molecules which are catenated to the specific target sequences (see e.g., Nilsson, et al., 1994. Science 265: 2085-2088). The resulting probes are suitable for the simultaneous analysis of many gene sequences both due to their specificity and selectivity for gene sequence variants (see e.g., Lizardi, et al., 1998. Nat. Genet. 19: 225-232; Nilsson, et al., 1997. Nat. Genet. 16: 252-255) and due to the fact that the resulting reaction products remain localized to the specific target sequences. Moreover, intramolecular ligation of many different probes is expected to be less susceptible to non-specific cross-reactivity than multiplex PCR-based methodologies where non-cognate pairs of primers can give rise to irrelevant amplification products (see e.g., Landegren and Nilsson, 1997. Ann Med 29: 585-590).

A starting library can be constructed comprising either single-stranded or double-stranded nucleic acid molecules, provided that the nucleic acid sequence includes a region that, if present in the library, is available for annealing, or can be made available for annealing, to an anchor primer sequence. For example, when used as a template for rolling circle amplification, a region of a double-stranded template needs to be at least transiently single-stranded in order to act as a template for extension of the anchor primer.

Library templates can include multiple elements, including, but not limited to, one or more regions that are complementary to the anchor primer. For example, the template libraries may include a region complementary to a sequencing primer, a control nucleotide region, and an insert sequence comprised of the sequencing template to be subsequently characterized. As is explained in more detail below, the control nucleotide region is used to calibrate the relationship between the amount of byproduct and the number of nucleotides incorporated. As utilized herein the term “complement” refers to nucleotide sequences that are able to hybridize to a specific nucleotide sequence to form a matched duplex.

In one embodiment, a library template includes: (i) two distinct regions that are complementary to the anchor primer, (ii) one region homologous to the sequencing primer, (iii) one optional control nucleotide region, (iv) an insert sequence of, e.g., 30-500, 50-200, or 60-100 nucleotides, that is to be sequenced. The template can, of course, include two, three, or all four of these features.

The template nucleic acid can be constructed from any source of nucleic acid, e.g., any cell, tissue, or organism, and can be generated by any art-recognized method. Suitable methods include, e.g., sonication of genomic DNA and digestion with one or more restriction endonucleases (RE) to generate fragments of a desired range of lengths from an initial population of nucleic acid molecules. Preferably, one or more of the restriction enzymes have distinct four-base recognition sequences. Examples of such enzymes include, e.g., Sau3A1, MspI, and TaqI. Preferably, the enzymes are used in conjunction with anchor primers having regions containing recognition sequences for the corresponding restriction enzymes. In some embodiments, one or both of the adapter regions of the anchor primers contain additional sequences adjoining known restriction enzyme recognition sequences, thereby allowing for capture or annealing to the anchor primer of specific restriction fragments of interest to the anchor primer. In other embodiments, the restriction enzyme is used with a type IIS restriction enzyme.

Alternatively, template libraries can be made by generating a complementary DNA (cDNA) library from RNA, e.g., messenger RNA (mRNA). The cDNA library can, if desired, be further processed with restriction endonucleases to obtain a 3′ end characteristic of a specific RNA, internal fragments, or fragments including the 3′ end of the isolated RNA. Adapter regions in the anchor primer may be complementary to a sequence of interest that is thought to occur in the template library, e.g., a known or suspected sequence polymorphism within a fragment generated by endonuclease digestion.

In one embodiment, an indexing oligonucleotide can be attached to members of a template library to allow for subsequent correlation of a template nucleic acid with a population of nucleic acids from which the template nucleic acid is derived. For example, one or more samples of a starting DNA population can be fragmented separately using any of the previously disclosed methods (e.g., restriction digestion, sonication). An indexing oligonucleotide sequence specific for each sample is attached to, e.g., ligated to, the termini of members of the fragmented population. The indexing oligonucleotide can act as a region for circularization, amplification and, optionally, sequencing, which permits it to be used to index, or code, a nucleic acid so as to identify the starting sample from which it is derived.

Distinct template libraries made with a plurality of distinguishable indexing primers can be mixed together for subsequent reactions. Determining the sequence of the member of the library allows for the identification of a sequence corresponding to-the indexing oligonucleotide. Based on this information, the origin of any given fragment can be inferred.

Libraries of nucleic acids are annealed to anchor primer sequences using recognized techniques (see, e.g., Hatch, et al., 1999. Genet. Anal. Biomol. Engineer. 15: 35-40; Kool, U.S. Pat. No. 5,714,320 and Lizardi, U.S. Pat. No. 5,854,033). In general, any procedure for annealing the anchor primers to the template nucleic acid sequences is suitable as long as it results in formation of specific, i.e., perfect or nearly perfect, complementarity between the adapter region or regions in the anchor primer sequence and a sequence present in the template library.

A number of in vitro nucleic acid amplification techniques may be utilized to extend the anchor primer sequence. The size of the amplified DNA preferably is smaller than the size of the anchor pad and also smaller than the distance between anchor pads.

The amplification is typically performed in the presence of a polymerase, e.g., a DNA or RNA-directed DNA polymerase, and one, two, three, or four types of nucleotide triphosphates, and, optionally, auxiliary binding proteins. In general, any polymerase capable of extending a primed 3′—OH group can be used a long as it lacks a 3′ to 5′ exonuclease activity. Suitable polymerases include, e.g., the DNA polymerases from Bacillus stearothermophilus, Thermus acquaticus, Pyrococcus furiosis, Thermococcus litoralis, and Thermus thermophilus, bacteriophage T4 and T7, and the E. coli DNA polymerase I Klenow fragment. Suitable RNA-directed DNA polymerases include, e.g., the reverse transcriptase from the Avian Myeloblastosis Virus, the reverse transcriptase from the Moloney Murine Leukemia Virus, and the reverse transcriptase from the Human Immunodeficiency Virus-I.

A number of in vitro nucleic acid amplification techniques have been described. These amplification methodologies may be differentiated into those methods: (i) which require temperature cycling—polymerase chain reaction (PCR) (see e.g., Saiki, et al., 1995. Science 230: 1350-1354), ligase chain reaction (see e.g., Barany, 1991. Proc. Natl. Acad Sci. USA 88: 189-193; Barringer, et al., 1990. Gene 89: 117-122) and transcription-based amplification (see e.g., Kwoh, et al., 1989. Proc. Natl. Acad Sci. USA 86:1173-1177) and (ii) isothermal amplification systems—self-sustaining, sequence replication (see e.g., Guatelli, et al, 1990. Proc. Natl. Acad Sci. USA 87: 1874-1878); the Qβ replicase system (see e.g., Lizardi, et al., 1988. BioTechnology 6: 1197-1202); strand displacement amplification Nucleic Acids Res. 1992 Apr 11;20(7):1691-6.; and the methods described in PNAS 1992 Jan 1;89(1):392-6; and NASBA J Virol Methods. 1991 Dec;35(3):273-86.

Isothermal amplification also includes rolling circle-based amplification (RCA). RCA is discussed in, e.g., Kool, U.S. Pat. No. 5,714,320 and Lizardi, U.S. Pat. No. 5,854,033, Hatch, et al., 1999. Genet. Anal. Biomol. Engineer. 15: 35-40. The result of the RCA is a single DNA strand extended from the 3′ terminus of the anchor primer (and thus is linked to the solid support matrix) and including a concatamer containing multiple copies of the circular template annealed to a primer sequence. Typically, 1,000 to 10,000 or more copies of circular templates, each having a size of, e.g., approximately 30-500, 50-200, or 60-100 nucleotides size range, can be obtained with RCA.

In vivo, RCR is utilized in several biological systems. For example, the genome of several bacteriophage are single-stranded, circular DNA. During replication, the circular DNA is initially converted to a duplex form, which is then replicated by the aforementioned rolling-circle replication mechanism. The displaced terminus generates a series of genomic units that can be cleaved and inserted into the phage particles. Additionally, the displaced single-strand of a rolling-circle can be converted to duplex DNA by synthesis of a complementary DNA strand. This synthesis can be used to generate the concatemeric duplex molecules required for the maturation of certain phage DNAs. For example, this provides the principle pathway by which λ bacteriophage matures. RCR is also used in vivo to generate amplified rDNA in Xenopus oocytes, and this fact may help explain why the amplified rDNA is comprised of a large number of identical repeating units. In this case, a single genomic repeating unit is converted into a rolling-circle. The displaced terminus is then converted into duplex DNA which is subsequently cleaved from the circle so that the two termini can be ligated together so as to generate the amplified circle of rDNA.

Through the use of the RCA reaction, a strand may be generated which represents many tandem copies of the complement to the circularized molecule. For example, RCA has recently been utilized to obtain an isothermal cascade amplification reaction of circularized padlock probes in vitro in order to detect single-copy genes in human genomic DNA samples (see Lizardi, et al., 1998. Nat. Genet. 19: 225-232). In addition, RCA has also been utilized to detect single DNA molecules in a solid phase-based assay, although difficulties arose when this technique was applied to in situ hybridization (see Lizardi, et al., 1998. Nat. Genet. 19: 225-232).

If desired, RCA can be performed at elevated temperatures, e.g., at temperatures greater than 37° C., 42° C., 45° C., 50° C., 60° C., or 70° C. In addition, RCA can be performed initially at a lower temperature, e.g., room temperature, and then shifted to an elevated temperature. Elevated temperature RCA is preferably performed with thermostable nucleic acid polymerases and with primers that can anneal stably and with specificity at elevated temperatures.

RCA can also be performed with non-naturally occurring oligonucleotides, e.g., peptide nucleic acids. Further, RCA can be performed in the presence of auxiliary proteins such as single-stranded binding proteins.

The development of a method of amplifying short DNA molecules which have been immobilized to a solid support, termed RCA has been recently described in the literature (see e.g., Hatch, et al., 1999. Genet. Anal. Biomol. Engineer. 15: 35-40; Zhang, et al., 1998. Gene 211: 277-85; Baner, et al., 1998. Nucl. Acids Res. 26: 5073-5078; Liu, et al, 1995. J. Am. Chem. Soc. 118: 1587-1594; Fire and Xu, 1995. Proc. Natl. Acad Sci. USA 92: 4641-4645; Nilsson, et al., 1994. Science 265: 2085-2088). RCA targets specific DNA sequences through hybridization and a DNA ligase reaction. The circular product is then subsequently used as a template in a rolling circle replication reaction.

RCA driven by DNA polymerase can replicate circularized oligonucleotide probes with either linear or geometric kinetics under isothermal conditions. In the presence of two primers (one hybridizing to the + strand, and the other, to the − strand of DNA), a complex pattern of DNA strand displacement ensues which possesses the ability to generate 1×10⁹ or more copies of each circle in a short period of time (i.e., less-than 90 minutes), enabling the detection of single-point mutations within the human genome. Using a single primer, RCA generates hundreds of randomly-linked copies of a covalently closed circle in several minutes. If solid support matrix-associated, the DNA product remains bound at the site of synthesis, where it may be labeled, condensed, and imaged as a point light source. For example, linear oligonucleotide probes, which can generate RCA signals, have been bound covalently onto a glass surface. The color of the signal generated by these probes indicates the allele status of the target, depending upon the outcome of specific, target-directed ligation events. As RCA permits millions of individual probe molecules to be counted and sorted, it is particularly amenable for the analysis of rare somatic mutations. RCA also shows promise for the detection of padlock probes bound to single-copy genes in cytological preparations.

In addition, a solid-phase RCA methodology has also been developed to provide an effective method of detecting constituents within a solution. Initially, a recognition step is used to generate a complex h a circular template is bound to a surface. A polymerase enzyme is then used to amplify the bound complex. RCA uses small DNA probes that are amplified to provide an intense signal using detection methods, including the methods described in more detail below.

Other examples of isothermal amplification systems include, e.g., (i) self-sustaining, sequence replication (see e.g., Guatelli, et al., 1990. Proc. Natl. Acad. Sci. USA 87: 1874-1878), (ii) the Qβ replicase system (see e.g., Lizardi, et al., 1988. BioTechnology 6: 1197-1202), and (iii) nucleic acid sequence-based amplification (NASBA™; see Kievits, et al., 1991. J. Virol. Methods 35: 273-286).

Amplification of a nucleic acid template as described above results in multiple copies of a template nucleic acid sequence covalently linked to an anchor primer. In one embodiment, a region of the sequence product is determined by annealing a sequencing primer to a region of the template nucleic acid, and then contacting the sequencing primer with a DNA polymerase and a known nucleotide triphosphate, i.e., dATP, dCTP, dGTP, dTTP, or an analog of one of these nucleotides. The sequence can be determined by detecting a sequence reaction byproduct, as is described below.

The sequence primer can be any length or base composition, as long as it is capable of specifically annealing to a region of the amplified nucleic acid template. No particular structure for the sequencing primer is required so long as it is able to specifically prime a region on the amplified template nucleic acid. Preferably, the sequencing primer is complementary to a region of the template that is between the sequence to be characterized and the sequence hybridizable to the anchor primer. The sequencing primer is extended with the DNA polymerase to form a sequence product. The extension is performed in the presence of one or more types of nucleotide triphosphates, and if desired, auxiliary binding proteins.

The method comprises the steps of: (a) introducing the template nucleic acid polymer into a polymerization environment in which the nucleic acid polymer will act as a template polymer for the synthesis of a complementary nucleic acid polymer when nucleotides are added; (b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released; (c) separately recovering each of the feedstocks from the polymerization environment; and (d) measuring the amount of inorganic pyrophosphate by utilizing an ATP generating polypeptide-ATP converting polypeptide fusion protein in each of the recovered feedstocks to determine the identity of each nucleotide in the complementary polymer and thus the sequence of the template polymer.

The sequence primer can be any length or base composition, as long as it is capable of specifically annealing to a region of the amplified nucleic acid template. No particular structure is required for the sequencing primer so long as it is able to specifically prime a region on the amplified template nucleic acid. Preferably, the sequencing primer is complementary to a region of the template that is between the sequence to be characterized and the sequence hybridizable to the anchor primer. The sequencing primer is extended with the DNA polymerase to form a sequence product. The extension is performed in the presence of one or more types of nucleotide triphosphates, and if desired, auxiliary binding proteins.

This invention also includes a method wherein the amount of inorganic pyrophosphate is measured by (a) adding adenosine-5′-phosphosulfate to the feedstock; combining the recovered feedstock containing adenosine-5′-phosphosulfate with an ATP generating polypeptide-ATP converting polypeptide fusion protein such that any inorganic pyrophosphate in the recovered feedstock and the adenosine-5′-phosphosulfate will first react to the form ATP and sulfate and then react with luciferin in the presence of oxygen such that the ATP is consumed to produced AMP, inorganic pyrophosphate, carbon dioxide and light; and (b) measuring the amount of light produced. In a preferred embodiment, the template polymer and ATP generating polypeptide-ATP converting polypeptide fusion protein are immobilized on a solid support.

The invention also includes a method for determining the nucleic acid sequence in a template nucleic acid polymer, comprising: (a) introducing the template nucleic acid polymer into a polymerization environment in which the nucleic acid polymer will act as a template polymer for the synthesis of a complementary nucleic acid polymer when nucleotides are added; (b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released; (c) separately recovering each of the feedstocks from the polymerization environment; and (d) measuring the amount of PPi with a thermostable sulfurylase and a luciferase in each of the recovered feedstocks to determine the identity of each nucleotide in the complementary polymer and thus the sequence of the template polymer. In one embodiment, the thermostable sulfurylase and the luciferase are joined in a fusion protein. In another embodiment, the thermostable sulfurylase is joined to an affinity tag.

The invention further provides a method for sequencing a nucleic acid, the method comprising: (a) providing one or more nucleic acid anchor primers; (b) providing a plurality of single-stranded circular nucleic acid templates disposed within a plurality of cavities on a planar surface, each cavity forming an analyte reaction chamber, wherein the reaction chambers have a center to center spacing of between 5 to 200; (c) annealing an effective amount of the nucleic acid anchor primer to at least one of the single-stranded circular templates to yield a primed anchor primer-circular template complex; (d) combining the primed anchor primer-circular template complex with a polymerase to form an extended anchor primer covalently linked to multiple copies of a nucleic acid complementary to the circular nucleic acid template; (e) annealing an effective amount of a sequencing primer to one or more copies of said covalently linked complementary nucleic acid; (f) extending the sequencing primer with a polymerase and a predetermined nucleotide triphosphate to yield a sequencing product and, if the predetermined nucleotide triphosphate is incorporated onto the 3′ end of said sequencing primer, a sequencing reaction byproduct; and (g) identifying the sequencing reaction byproduct with the use of a thermostable sulfurylase and a luciferase, thereby determining the sequence of the nucleic acid. In one embodiment, the thermostable sulfurylase and the luciferase are joined in a fusion protein. In another embodiment, the thermostable sulfurylase is joined to an affinity tag.

Also included in the invention is a method for sequencing a nucleic acid, the method comprising: (a) providing at least one nucleic acid anchor primer; (b) providing a plurality of single-stranded circular nucleic acid templates in an array having at least 400,000 discrete reaction sites; (c) annealing a first amount of the nucleic acid anchor primer to at least one of the single-stranded circular templates to yield a primed anchor primer-circular template complex; (d) combining the primed anchor primer-circular template complex with a polymerase to form an extended anchor primer covalently linked to multiple copies of a nucleic acid complementary to the circular nucleic acid template; (e) annealing a second amount of a sequencing primer to one or more copies of the covalently linked complementary nucleic acid; (f) extending the sequencing primer with a polymerase and a predetermined nucleotide triphosphate to yield a sequencing product and, when the predetermined nucleotide triphosphate is incorporated onto the 3′ end of the sequencing primer, to yield a sequencing reaction byproduct; and (g) identifying the sequencing reaction byproduct with the use of a thermostable sulfurylase and a luciferase, thereby determining the sequence of the nucleic acid at each reaction site that contains a nucleic acid template. In one embodiment, the thermostable sulfurylase and the luciferase are joined in a fusion protein. In another embodiment, the thermostable sulfurylase is joined to an affinity tag.

The invention also includes a method of determining the base sequence of a plurality of nucleotides on an array, the method comprising: (a) providing a plurality of sample DNAs, each disposed within a plurality of cavities on a planar surface, each cavity forming an analyte reaction chamber, wherein the reaction chambers have a center to center spacing of between 5 to 200 μm, (b) adding an activated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates; (c) detecting whether or not the nucleoside 5′-triphosphate precursor was incorporated into the primer strands through detection of a sequencing byproduct with a thermostable sulfurylase and luciferase, thus indicating that the unpaired nucleotide residue of the template has a nitrogenous base composition that is complementary to that of the incorporated nucleoside 5′-triphosphate precursor; and (d) sequentially repeating steps (b) and (c), wherein each sequential repetition adds and, detects the incorporation of one type of activated nucleoside 5′-triphosphate precursor of known nitrogenous base composition; and (e) determining the base sequence of the unpaired nucleotide residues of the template in each reaction chamber from the sequence of incorporation of said nucleoside precursors. In one embodiment, the thermostable sulfurylase and the luciferase are joined in a fusion protein. In another embodiment, the thermostable sulfurylase is joined to an affinity tag.

The invention will be further illustrated in the following non-limiting examples. There are several abbreviations which will be used in the following examples: FUS stands for fusion gene, S stands for sulfurylase, L stands for luciferase, TL stands for thermostable luciferase, X stands for XhoI, H stands for HindIII, N stands for NotI and B stands for BamHI. For example, FUS-L/S X F means a primer for the fusion gene, luciferase-sulfurylase Xho Forward and so forth. Primers 1 through 6 are for the L or TL to S fusions and primers 7 through 13 are for the S to L or TL fusions.

### EXAMPLES

### Example 1

### Cloning Strategy for Obtaining the Bst Sulfurylase Gene

Gene specific primers, which incorporated restriction site linkers, were designed based on the sequence for a putative ATP sulfurylase from Bacillus stearothermophilus in ERGO, a curated database of genomic DNA made available on the World Wide Web by Integrated Genomics which included the Bacillus stearothermophilus Genome Sequencing Project at the University of Oklahoma (NSF Grant #EPS-9550478). The forward primer utilized was 5′-CCC TTC TGC AGC ATG AGC GTA AGC ATC CCG CAT GGC GGC ACA TTG-3′ (SEQ ID NO: 7) and the reverse primer used was 5′-CCC GTA AGC TTT TAG CGC GCT GAC GGG GCG ACC GTT TCG CGT TCT TG-3′ (SEQ ID NO:8). The reaction mix for PCR amplification contained 5.0 uL 10X polymerase buffer (Clontech, Cat. #8714), 2.0 uL 5 M betaine (Sigma, Cat. #B0300), 1.0 uL dNTP mix (10 mM each dATP, dCTP, dGTP, dTTP), 0.8 uL Advantage 2 polymerase (Clontech, Cat. #8714), 0.2 uL Advantage-HF 2 polymerase (Clontech, Cat. #K1914), 10 pmol forward primer, 10 pmol reverse primer, 100 ng (or less) Bst genomic DNA (ATCC, Cat. #12980D), and enough distilled water to make total volume of 50 uL. As little as 1 ng Bst genomic DNA was sufficient to yield PCR product. The PCR amplification of Bst ATP sulfurylase gene from genomic DNA consisted of an initial step at 96° C. for 3 min, then 35 cycles of 96° C. for 15 sec, 60° C. for 30 sec, 72° C. for 6 min, a finishing step at 72° C. for 10 min and finally 14° C. until removal. The PCR product was cleaned using QIAquick PCR Purification Kit (QIAGEN).

### Example 2

### Cloning Strategy for Obtaining the Sulfurylase-Luciferase Fusion Protein

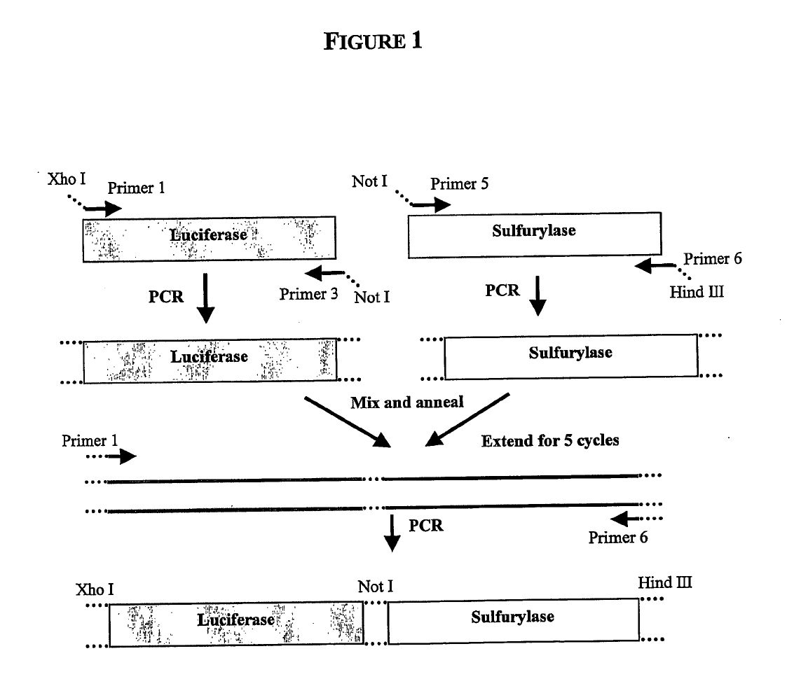

All chemicals were purchased from Sigma unless noted otherwise. Racemically pure D-luciferin was ordered from Pierce. The assay buffer for measuring ATP sulfurylase and luciferase activities contained Taq polymerase. A polymerase chain reaction (PCR)-mediated approach was utilized to link the open reading frames (ORFs) of luciferase and sulfurylase. The cloning strategy is outlined in FIG. 1. Briefly, it involved the amplification of luciferase and sulfurylase ORFs by PCR, using primers that contain convenient restriction sites (XhoI and HindIII) to clone the fusion gene into an expression vector, in-frame and, the design of a rare restriction site (Not I) at the junction of the two polypeptides so that other versions of luciferase, such as thermostable luciferase (TL), and sulfurylase can be conveniently swapped to obtain either sulfurylase-luciferase (S-L) or luciferase-sulfurylase (L-S) fusion proteins. A Not I site was used to fuse the variable heavy chain of antibodies to luciferase to generate a viable fusion protein. These primers were also designed in such a way that the primers that form part of the junction of the two ORFs contain sufficient overlapping regions of nucleotides. For example, the 5′ end of FUS-L/S Not R contains deoxynucleotides in an anti-parallel orientation that encode the N-terminal 10 amino acids of yeast sulfurylase. Thus, a PCT product generated using this primer would anneal to the 5′ end of yeast sulfurylase ORF and would generate the fusion protein, L-S.

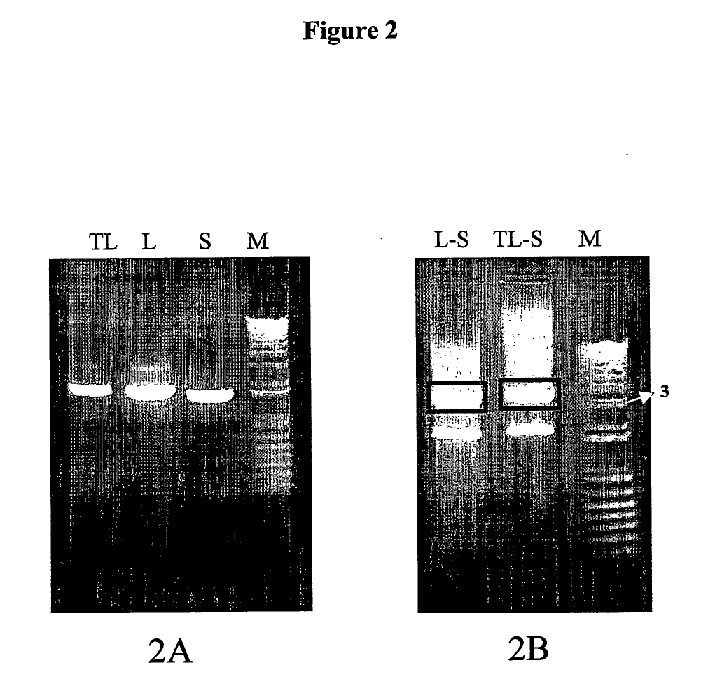

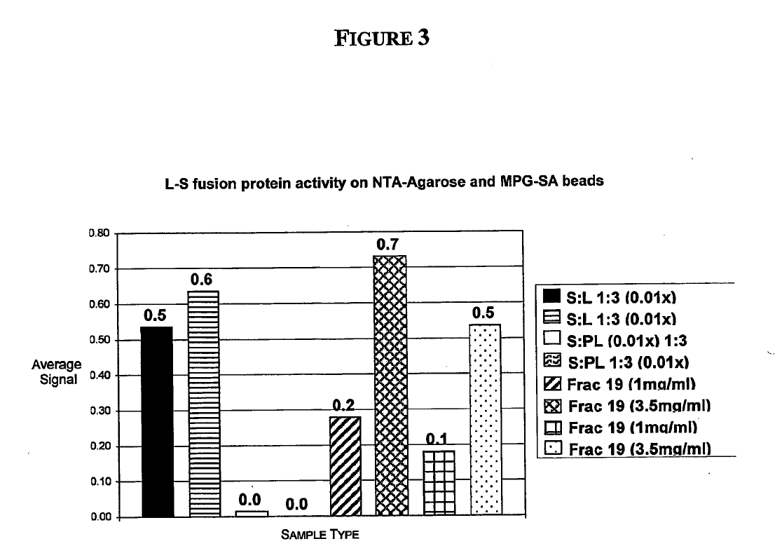

The products in boxes were obtained by PCR as elaborated in FIG. 2. As shown in FIG. 3, the PCT products were subjected to electrophoresis. The PCR products were then purified, digested with Xho I and Hind III and subcloned into Xho I/Hind III digested pRSETA-BCCP. pRSETA-BCCP is a derivative of pRSET A (Invitrogen) in which the sequence between NheI and BamHI restriction sites has been replaced by the portion of the biotin carboxyl carrier protein BCCP) gene from E. coli (GenBank accession #M80458) that codes for residues 87-165. The 87- amino acid BCCP domain was obtained by PCR and cloned into the NheI and Bam HI sites of pRSETA to obtain pRSETA-BCCP. The ligated fusion protein and pRSETA-BCCP were transformed into BL21DE3 and TOP10 cells. BL21DE3 cells yielded colonies for L-S and TOP10 cells yielded colonies for TL-S.

The following list of primers was used to construct the fusion proteins:

| | | | |
|---|---|---|---|
| | | | SEQ |
| PRIMER | | | ID |
| NO | TITLE | NUCLEIC ACID SEQUENCE | NO |

| | | | |
|---|---|---|---|
| 1 | FUS-L/S X F | CCCC CTC GAG ATC CAA ATG GAA GAC GCC AAA | 9 |
| | | AAC ATA AAG AAA GGC CC | |
| 2 | FUS-TL/S X | CCCC CTC GAG ATC CAA ATG GCT GAC AAA AAC | 10 |
| | F | ATC CTG TAT GGC CC | |
| 3 | FUS-L/S Not | TTG TAG AAT ACC ACC GTG AGG AGC AGG CAT | 11 |
| | R | AGC GGC CGC CAA TTT GGA CTT TCC GCC CTT CTT | |
| | | GGC C | |
| 4 | FUS-TL/S | TTG TAG AAT ACC ACC GTG AGG AGC AGG CAT | 12 |
| | Not R | AGC GGC CGC ACC GTT GGT GTG TTT CTC GAA CAT | |
| | | C | |
| 5 | FUS-S-Not F | GCG GCC GCT ATG CCT GCT CCT CAC GGT GGT ATT | 13 |
| | | CTA C | |
| 6 | FUS-S-Hind | CCCC AAG CTT TTA AAA TAC AAA AAA GCC ATT | 14 |
| | III R | GTC TTC CAA GAA TAG GAC | |
| 7 | FUS-S/L B F | CCCC GGA TCC ATC CAA ATG CCT GCT CCT CAC | 15 |
| | | GGT GGT ATT CTA CAA GAC | |
| 8 | FUS-S/L R | GGGCCTTTCTTTATGTTTTTGGCGTCTTCCAT AGC | 16 |
| | | GGC CGC AAA TAC AAA AAA GCC ATT GTC | |
| 9 | FUS-L-F | GCG GCC GCT ATG GAA GAC GCC AAA AAC ATA | 17 |
| | | AAG AAA GGC CC | |
| 10 | FUS-L-N-R | CCCC CCA TGG TTA CAA TTT GGA CTT TCC GCC | 18 |
| | | CTT CTT GGC C | |
| 11 | FUS-S/TL R | GG GCC ATA CAG GAT GTT TTT GTC AGC CAT AGC | 19 |
| | | GGC CGC AAA TAC AAA AAA GCC ATT GTC | |
| 12 | FUS-TL-F | GCG GCC GCT ATG GCT GAC AAA AAC ATC CTG | 20 |
| | | TAT GGC CC | |
| 13 | FUS-TL-H-R | CCCC AAG CTT CTA ACC GTT GGT GTG TTT CTC | 21 |
| | | GAA CAT CTG ACG C | |

These primers were utilized to perform PCR. The following PCR condition was used.

PCR condition
96° C. for 3:00; 96° C. for 0:15; 76° C. for 0:30; −1° C. per cycle; 72° C. for 6:00;
For 15 cycles; 96° C. for 0:15; 60° C. for 0:30; 72° C. for 6:00;
For 29 cycles; 72° C. for 10:00;
14° C. forever

### Example 3

### Cloning of the His6-BCCP Bst ATP Sulfurylase Fusion Protein

The Bst-affinity tagged fusion construct is a derivative of pRSETA in which the NheI-XhoI fragment has been replaced by the BCCP domain and the ATP sulfurylase is inserted after the BCCP domain.

Briefly, the BstSulf PCR product, as described in Example 1, was double-digested with PstI and HindIII, isolated on a 1% agarose/TAE gel, purified using QIAEXII (QIAGEN) and ligated into the large PstI/HindIII fragment of pRSETA-BCCP using the Quick Ligation Kit from NEB according to manufacturer's instructions. As mentioned in Example 2, pRSETA-BCCP is a derivative of pRSET A (Invitrogen) in which the sequence between NheI and BamHI restriction sites has been replaced by the portion of the biotin carboxyl carrier protein (BCCP) gene from E. coli (GenBank accession #M80458) that codes for residues 87-165. 2 uL ligation reaction was used to transform 50 uL TOP10 competent cells (Invitrogen) and plated on LB-Ap plates. Sequencing of plasmid insert from ten clones was used to determine the consensus sequence for the ATP sulfurylase gene from ATCC 12980.

The plasmid pRSETA-BCCP-BstSulf was transformed into the E. coli expression host BL21(DE3)pLysS (Novagen) and the induction expression of BstHBSulf was carried out according to the manufacturer's instructions. The cells were harvested and stored as frozen pellets. The pellets were lysed using BugBuster plus Benzonase according to manufacturer's instructions and protein was purified on a 20 mL column packed with Chelating Sepharose Fast Flow (Amersham, Cat. #17-0575-02) and charged with nickel (II). Protein was eluted using a 0-500 mM imidazole gradient. Analysis by SDS-PAGE showed a single band of the correct size.

### Example 4

### Binding enzymes to beads

The BCCP domain enables the E. coli to add a single biotin molecule onto a specific lysine residue. Hence these fusion proteins can be bound to solid supports that contain streptavidin. TL-S was successfully cloned into a TA vector. 25 μl of MPG-Streptavidin (CPG, Inc.) or Nickel-agarose (Qiagen) were taken in a 1.5 ml tube and placed on a magnet. The supernatant was removed and the beads were resuspended in 25 μg of His6-BCCP-sulfurylase and 75 μg of His6-BCCP-luciferase. To test the fusion protein, 100 μl of dialyzed fusion protein was bound to the 25 μl of beads. The beads were allowed to mix at room temperature for 1 hr, washed with assay buffer (25 mM Tricine (pH 7.8), 5 mM MgAcetate, 1 mM DTT, 1 mM EDTA, and 1 mg/ml BSA) and assayed for enzyme activities with 1 mM PPi, 4 mM APS and 300 mM D-luciferin. With the nickel-agarose beads, the EDTA was omitted from the assay buffer.

As shown in FIG. 4, these fusion proteins displayed activity on both the NTA-Agarose and MPG-SA beads. S:L 1:3 represents sulfurylase and luciferase bound individually to beads in a 1:3 ratio. Ni-Ag and MPG-SA are nickel-agarose and MPG-Streptavidin beads, respectively. PL is Promega luciferase, which does not have a polyhistidine or a biotin tag on it and hence serves as a negative control. Fraction 19 contains the fusion protein and is active on both kinds of beads. This suggests that the fusion protein was synthesized with a poly-histidine tag and a biotin molecule on the BCCP domain of the fusion protein.

1. A fusion protein comprising an ATP generating polypeptide bound to a polypeptide which converts ATP to an entity that is detectable.

2. The fusion protein of claim 1 wherein the ATP generating polypeptide is selected from the group consisting of ATP sulfurylase, hydrolase and ATP synthase.

3. The fusion protein of claim 2 wherein the ATP sulfurylase is a thermostable sulfurylase comprising the nucleotide sequence of SEQ ID NO:1.

4. The fusion protein of claim 3 wherein the nucleotide sequence encodes the polypeptide sequence of SEQ ID NO:2.

5. The fusion protein of claim 3 wherein the thermostable sulfurylase is active at room temperature.

6. The fusion protein of claim 2 wherein the ATP sulfurylase is from a thermophile.

7. The fusion protein of claim 6 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

8. The fusion protein of claim 1 wherein the ATP generating polypeptide and ATP converting polypeptide are from a eukaryote or a prokaryote.

9. The fusion protein of claim 8 wherein the eukaryote is selected from the group consisting of animal, plant, fungus and yeast.

10. The fusion protein of claim 9 wherein the animal is selected from the group consisting of mammal, rodent, insect, worm, mollusk, reptile, bird and amphibian.

11. The fusion protein of claim 9 wherein the plant is selected from the group consisting of Arabidopsis thaliana, Brassica napus, Allium sativum, Amaranthus caudatus, Hevea brasiliensis, Hordeum vulgare, Lycopersicon esculentum, Nicotiana tabacum, Oryza sativum, Pisum sativum, Populus trichocarpa, Solanum tuberosum, Secale cereale, Sambucus nigra, Ulmus americana or Triticum aestivum.

12. The fusion protein of claim 9 wherein the fungus is Penicillum chrysogenum, Stachybotrys chartarum, Aspergillus fumigatus, Podospora anserina, Trichoderma reesei and Riftia pachyptila.

13. The fusion protein of claim 9 wherein the yeast is Saccharomyces cerevisiae, Candida tropicalis, Candida lypolitica, Candida utilis, Kluyveromyces lactis, Schizosaccharomyces pombe, Yarrowia lipolytica, Candida spp., Pichia spp. and Hansenula spp..

14. The fusion protein of claim 8 wherein the prokaryote is bacteria or archaea.

15. The fusion protein of claim 14 wherein the bacteria selected from the group consisting of E. coli, B. subtilis, Streptococcus gordonii, flavobacteria and green sulfur bacteria.

16. The fusion protein of claim 14 wherein the archaea is selected from the group consisting of Sulfolobus, Thermococcus, Methanobacterium, Halococcus, Halobacterium and Methanococcus jannaschii.

17. The fusion protein of claim 1 wherein the detectable entity is selected from the group consisting of chemiluminescence, bioluminescence and fluorescence.

18. The fusion protein of claim 1 wherein the ATP converting polypeptide is selected from the group consisting of luciferase, ecto-nucleoside diphosphate kinase and ATPase.

19. The fusion protein of claim 18 wherein the luciferase is selected from the group consisting of Photinus pyralis, Pyroplorus plagiophihalamus (Coleoptera), Luciola cruciata and Luciola lateralis.

20. The fusion protein of claim 1 which further comprises an affinity tag.

21. The fusion protein of claim 20 wherein the affinity tag is selected from the group consisting of N-terminal poly-histidine, BCCP, protein A, glutathione S transferase, substance P and streptavidin binding peptide.

22. The fusion protein of claim 1 wherein the polypeptides are joined by a linker.

23. The fusion protein of claim 22 wherein the linker is an ala-ala-ala linker.

24. The fusion protein of claim 1 wherein the ATP generating polypeptide is N-terminal to the ATP converting polypeptide.

25. The fusion protein of claim 1 wherein the ATP converting polypeptide is N-terminal to the ATP generating polypeptide.

26. An isolated nucleic acid molecule comprising a nucleic acid sequence selected from the group consisting of SEQ ID NOs:1, 3 and 5.

27. An isolated polypeptide comprising an amino acid sequence selected from the group consising of SEQ ID NOs:2, 4 and 6.

28. A fusion protein comprising a sulfurylase polypeptide bound to a luciferase polypeptide and at least one affinity tag.

29. The fusion protein of claim 28 wherein the fusion protein comprises the sequence of SEQ ID NO:4.

30. The fusion protein of claim 28 wherein the fusion protein is encoded by a nucleic acid comprising the sequence of SEQ ID NO:3.

31. A fusion protein comprising a thermostable sulfurylase bound to at least one affinity tag.

32. The fusion protein of claim 31 wherein the fusion protein comprises the sequence of SEQ ID NO: 6.

33. The fusion protein of claim 31 wherein the fusion protein is encoded by a nucleic acid comprising the sequence of SEQ ID NO:5.

34. A recombinant polynucleotide that comprises a coding sequence for a fusion protein having an ATP generating polypeptide sequence and an ATP converting polypeptide sequence.

35. The recombinant polynucleotide sequence of claim 34 wherein the ATP generating polypeptide is ATP sulfurylase.

36. The recombinant polynucleotide sequence of claim 34 wherein the ATP converting polypeptide is luciferase.

37. The recombinant polynucleotide of claim 34 wherein the ATP generating polypeptide is N-terminal to the ATP converting polypeptide.

38. The recombinant polynucleotide of claim 34 wherein the ATP converting polypeptide is N-terminal to the ATP generating polypeptide.

39. An expression vector for expressing a fusion protein, said vector comprising a coding sequence for a fusion protein having: (i) a regulatory sequence, (ii) a first polypeptide sequence of an ATP generating polypeptide and (iii) a second polypeptide sequence that converts ATP to an entity which is detectable.

40. The expression vector of claim 39 wherein the vector further comprises an affinity tag.

41. The expression vector of claim 39 wherein the ATP generating polypeptide is ATP sulfurylase.

42. The expression vector of claim 39 wherein the ATP converting polypeptide is luciferase.

43. The expression vector of claim 39 wherein the regulatory element is an enhancer or a promoter.

44. The expression vector of claim 43 wherein the promoter is a constitutive promoter or an inducible promoter.

45. A transformed host cell which contains the expression vector of claim 39.

46. The transformed host cell of claim 45 wherein the host cell is a eukaryotic cell.

47. The transformed host cell of claim 46 wherein the eukaryotic cell is human, rat or mouse.

48. The transformed host cell of claim 45 wherein the host cell is a prokaryotic cell.

49. The transformed host cell of claim 48 wherein the prokaryotic cell is bacteria.

50. A purified fusion protein expressed by cells transformed with an expression vector of claim 39.

51. The fusion protein of claim 1 bound to a mobile support.

52. The fusion protein of claim 51 wherein the fusion protein is attached by a covalent or non-covalent interaction.

53. The fusion protein of claim 52 wherein the fusion protein is attached by a linkage selected from the group consisting of a metal, a CO²⁺-hexahistidine complex, a Ni²⁺-hexahistidine complex, a biotin binding protein, a glutathione S-transferase/glutathione complex, a monoclonal antibody/antigen complex, a maltose binding protein/maltose complex and pluronic coupling.

54. The fusion protein of claim 53 wherein the biotin binding protein is selected from the group consisting of NEUTRAVIDIN™ modified avidin, streptavidin and avidin.

55. The fusion protein of claim 51 wherein the mobile support is selected from the group consisting of a bead, optical fiber and glass surface.

56. The fusion protein of claim 55 wherein the bead is a nickel-agarose bead or a MPG-Streptavidin bead.

57. The fusion protein of claim 51 wherein the fusion protein is bound to the mobile support in a 1:3 ratio of protein to mobile support.

58. The fusion protein of claim 51 wherein the fusion protein is a sulfurylase-luciferase fusion protein.

59. A method for determining the nucleic acid sequence in a template nucleic acid polymer, comprising:
(a) introducing the template nucleic acid polymer into a polymerization environment in which the nucleic acid polymer will act as a template polymer for the synthesis of a complementary nucleic acid polymer when nucleotides are added;
(b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released;
(c) separately recovering each of the feedstocks from the polymerization environment; and
(d) measuring the amount of PPi with an ATP generating polypeptide-ATP converting polypeptide fusion protein in each of the recovered feedstocks to determine the identity of each nucleotide in the complementary polymer and thus the sequence of the template polymer.

60. The method of claim 59 wherein the ATP generating polypeptide is selected from the group consisting of ATP sulfurylase, hydrolase and ATP synthase.

61. The method of claim 60 wherein the ATP sulfurylase is a thermostable sulfurylase.

62. The method of claim 60 wherein the ATP sulfurylase is from a thermophile.

63. The method of claim 62 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

64. The method of claim 59 wherein the ATP generating polypeptide and ATP converting polypeptide are from a eukaryote or a prokaryote.

65. The method of claim 64 wherein the eukaryote is selected from the group consisting of animal, plant, fungus and yeast.

66. The method of claim 59 wherein the ATP converting polypeptide is selected from the group consisting of luciferase, ecto-nucleoside diphosphate kinase and ATPase.

67. The method of claim 66 wherein the luciferase is selected from the group consisting of Photinus pyralis, Pyroplorus plagiophihalamus (Coleoptera), Luciola cruciata and Luciola lateralis.

68. The method of claim 59 which further comprises an affinity tag.

69. A method according to claim 59, wherein the amount of inorganic pyrophosphate is measured by
(a) adding adenosine-5′-phosphosulfate to the feedstock;
(b) combining the recovered feedstock containing adenosine-5′-phosphosulfate with an ATP generating polypeptide-ATP converting polypeptide fusion protein such that any inorganic pyrophosphate in the recovered feedstock and the adenosine-5′-phosphosulfate will react to the form ATP and sulfate;
(c) combining the ATP, sulfate, and said fusion protein-containing feedstock with luciferin in the presence of oxygen such that the ATP is consumed to produced AMP, inorganic pyrophosphate, carbon dioxide and light; and
(d) measuring the amount of light produced.

70. The method of claim 69 wherein the ATP generating polypeptide is selected from the group consisting of ATP sulfurylase, hydrolase and ATP synthase.

71. The method of claim 70 wherein the ATP sulfurylase is a thermostable sulfurylase.

72. The method of claim 70 wherein the ATP sulfurylase is from a thermophile.

73. The method of claim 72 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

74. The method of claim 69 wherein the ATP generating polypeptide and ATP converting polypeptide are from a eukaryote or a prokaryote.

75. The method of claim 74 wherein the eukaryote is selected from the group consisting of animal, plant, fungus and yeast.

76. The method of claim 69 wherein the ATP converting polypeptide is selected from the group consisting of luciferase, ecto-nucleoside diphosphate kinase and ATPase.

77. The method of claim 76 wherein the luciferase is selected from the group consisting of Photinus pyralis, Pyroplorus plagiophihalamus (Coleoptera), Luciola cruciata and Luciola lateralis.

78. The method of claim 69 which further comprises an affinity tag.

79. The method according to claim 59 wherein each feedstock comprises adenosine-5′-phosphosulfate and luciferin in addition to the selected nucleotide base, and the amount of inorganic pyrophosphate is determined by reacting the inorganic pyrophosphate is determined by reacting the inorganic pyrophosphate-containing feedstock with an ATP generating polypeptide-ATP converting polypeptide fusion protein thereby producing light in an amount proportional to the amount of inorganic pyrophosphate, and measuring the amount of light produced.

80. The method of claim 79 wherein the ATP generating polypeptide is selected from the group consisting of ATP sulfurylase, hydrolase and ATP synthase.

81. The method of claim 80 wherein the ATP sulfurylase is a thermostable sulfurylase.

82. The method of claim 80 wherein the ATP sulfurylase is from a thermophile.

83. The method of claim 82 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

84. The method of claim 79 wherein the ATP generating polypeptide and ATP converting polypeptide are from a eukaryote or a prokaryote.

85. The method of claim 84 wherein the eukaryote is selected from the group consisting of animal, plant, fungus and yeast.

86. The method of claim 79 wherein the ATP converting polypeptide is selected from the group consisting of luciferase, ecto-nucleoside diphosphate kinase and ATPase.

87. The method of claim 86 wherein the luciferase is selected from the group consisting of Photinus pyralis, Pyroplorus plagiophihalamus (Coleoptera), Luciola cruciata and Luciola lateralis.

88. The method of claim 79 which further comprises an affinity tag.

89. A method for sequencing a nucleic acid, the method comprising:
(a) providing one or more nucleic acid anchor primers;
(b) providing a plurality of single-stranded circular nucleic acid templates disposed within a plurality of cavities on a planar surface, each cavity forming an analyte reaction chamber, wherein the reaction chambers have a center to center spacing of between 5 to 200 μm;
(c) annealing an effective amount of the nucleic acid anchor primer to at least one of the single-stranded circular templates to yield a primed anchor primer-circular template complex;
(d) combining the primed anchor primer-circular template complex with a polymerase to form an extended anchor primer covalently linked to multiple copies of a nucleic acid complementary to the circular nucleic acid template;
(e) annealing an effective amount of a sequencing primer to one or more copies of said covalently linked complementary nucleic acid;
(f) extending the sequencing primer with a polymerase and a predetermined nucleotide triphosphate to yield a sequencing product and, if the predetermined nucleotide triphosphate is incorporated onto the 3′ end of said sequencing primer, a sequencing reaction byproduct; and
(g) identifying the sequencing reaction byproduct with the use of an ATP generating polypeptide-ATP converting polypeptide fusion protein, thereby determining the sequence of the nucleic acid.

90. The method of claim 89 wherein the ATP generating polypeptide is selected from the group consisting of ATP sulfurylase, hydrolase and ATP synthase.

91. The method of claim 90 wherein the ATP sulfurylase is a thermostable sulfurylase.

92. The method of claim 90 wherein the ATP sulfurylase is from a thermophile.

93. The method of claim 92 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

94. The method of claim 89 wherein the ATP generating polypeptide and ATP converting polypeptide are from a eukaryote or a prokaryote.

95. The method of claim 94 wherein the eukaryote is selected from the group consisting of animal, plant, fungus and yeast.

96. The method of claim 89 wherein the ATP converting polypeptide is selected from the group consisting of luciferase, ecto-nucleoside diphosphate kinase and ATPase.

97. The method of claim 96 wherein the luciferase is selected from the group consisting of Photinus pyralis, Pyroplorus plagiophihalamus (Coleoptera), Luciola cruciata and Luciola lateralis.

98. The method of claim 89 which further comprises an affinity tag.

99. A method for sequencing a nucleic acid, the method comprising:
(a) providing at least one nucleic acid anchor primer;
(b) providing a plurality of single-stranded circular nucleic acid templates in an array having at least 400,000 discrete reaction sites;
(c) annealing a first amount of the nucleic acid anchor primer to at least one of the single-stranded circular templates to yield a primed anchor primer-circular template complex;
(d) combining the primed anchor primer-circular template complex with a polymerase to form an extended anchor primer covalently linked to multiple copies of a nucleic acid complementary to the circular nucleic acid template;
(e) annealing a second amount of a sequencing primer to one or more copies of the covalently linked complementary nucleic acid;
(f) extending the sequencing primer with a polymerase and a predetermined nucleotide triphosphate to yield a sequencing product and, when the predetermined nucleotide triphosphate is incorporated onto the 3′ end of the sequencing primer, to yield a sequencing reaction byproduct; and
(g) identifying the sequencing reaction byproduct with the use of an ATP generating polypeptide-ATP converting polypeptide fusion protein, thereby determining the sequence of the nucleic acid at each reaction site that contains a nucleic acid template.

100. The method of claim 99 wherein the ATP generating polypeptide is selected from the group consisting of ATP sulfurylase, hydrolase and ATP synthase.

101. The method of claim 100 wherein the ATP sulfurylase is a thermostable sulfurylase.

102. The method of claim 100 wherein the ATP sulfurylase is from a thermophile.

103. The method of claim 102 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

104. The method of claim 99 wherein the ATP generating polypeptide and ATP converting polypeptide are from a eukaryote or a prokaryote.

105. The method of claim 104 wherein the eukaryote is selected from the group consisting of animal, plant, fungus and yeast.

106. The method of claim 99 wherein the ATP converting polypeptide is selected from the group consisting of luciferase, ecto-nucleoside diphosphate kinase and ATPase.

107. The method of claim 106 wherein the luciferase is selected from the group consisting of Photinus pyralis, Pyroplorus plagiophihalamus (Coleoptera), Luciola cruciata and Luciola lateralis.

108. The method of claim 99 which further comprises an affinity tag.

109. A method of determining the base sequence of a plurality of nucleotides on an array, the method comprising:
(a) providing a plurality of sample DNAs, each disposed within a plurality of cavities on a planar surface, each cavity forming an analyte reaction chamber, wherein the reaction chambers have a center to center spacing of between 5 to 200 μm,
(b) adding an activated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates;
(c) detecting whether or not the nucleoside 5′-triphosphate precursor was incorporated into the primer strands through detection of a sequencing byproduct with an ATP generating polypeptide-ATP converting polypeptide fusion protein, thus indicating that the unpaired nucleotide residue of the template has a nitrogenous base composition that is complementary to that of the incorporated nucleoside 5′-triphosphate precursor; and
(d) sequentially repeating steps (b) and (c), wherein each sequential repetition adds and, detects the incorporation of one type of activated nucleoside 5′-triphosphate precursor of known nitrogenous base composition; and
(e) determining the base sequence of the unpaired nucleotide residues of the template in each reaction chamber from the sequence of incorporation of said nucleoside precursors.

110. The method of claim 109 wherein the ATP generating polypeptide is selected from the group consisting of ATP sulfurylase, hydrolase and ATP synthase.

111. The method of claim 110 wherein the ATP sulfurylase is a thermostable sulfurylase.

112. The method of claim 110 wherein the ATP sulfurylase is from a thermophile.

113. The method of claim 112 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

114. The method of claim 109 wherein the ATP generating polypeptide and ATP converting polypeptide are from a eukaryote or a prokaryote.

115. The method of claim 114 wherein the eukaryote is selected from the group consisting of animal, plant, fungus and yeast.

116. The method of claim 109 wherein the ATP converting polypeptide is selected from the group consisting of luciferase, ecto-nucleoside diphosphate kinase and ATPase.

117. The method of claim 116 wherein the luciferase is selected from the group consisting of Photinus pyralis, Pyroplorus plagiophihalamus (Coleoptera), Luciola cruciata and Luciola lateralis.

118. The method of claim 109 which further comprises an affinity tag.

119. A method for determining the nucleic acid sequence in a template nucleic acid polymer, comprising:
(a) introducing a plurality of template nucleic acid polymers into a plurality of cavities on a planar surface, each cavity forming an analyte reaction chamber, wherein the reaction chambers have a center to center spacing of between 5 to 200 μm, each reaction chamber having a polymerization environment in which the nucleic acid polymer will act as a template polymer for the synthesis of a complementary nucleic acid polymer when nucleotides are added;
(b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released;
(c) detecting the formation of inorganic pyrophosphate with an ATP generating polypeptide-ATP converting polypeptide fusion protein to determine the identity of each nucleotide in the complementary polymer and thus the sequence of the template polymer.

120. The method of claim 119 wherein the ATP generating polypeptide is selected from the group consisting of ATP sulfurylase, hydrolase and ATP synthase.

121. The method of claim 120 wherein the ATP sulfurylase is a thermostable sulfurylase.

122. The method of claim 120 wherein the ATP sulfurylase is from a thermophile.

123. The method of claim 122 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

124. The method of claim 119 wherein the ATP generating polypeptide and ATP converting polypeptide are from a eukaryote or a prokaryote.

125. The method of claim 124 wherein the eukaryote is selected from the group consisting of animal, plant, fungus and yeast.

126. The method of claim 119 wherein the ATP converting polypeptide is selected from the group consisting of luciferase, ecto-nucleoside diphosphate kinase and ATPase.

127. The method of claim 126 wherein the luciferase is selected from the group consisting of Photinus pyralis, Pyroplorus plagiophihalamus (Coleoptera), Luciola cruciata and Luciola lateralis.

128. The method of claim 119 which further comprises an affinity tag.

129. A method of identifying the base in a target position in a DNA sequence of sample DNA, wherein:
(a) sample DNA is disposed within a plurality of cavities on a planar surface, each cavity forming an analyte reaction chamber, wherein the reaction chambers have a center to center spacing of between 5 to 200 μm, said DNA being rendered single stranded either before or after being disposed in the reaction chambers,
(b) an extension primer is provided which hybridizes to said immobilized single-stranded DNA at a position immediately adjacent to said target position;
(c) said immobilized single-stranded DNA is subjected to a polymerase reaction in the presence of a predetermined nucleotide triphosphate, wherein if the predetermined nucleotide triphosphate is incorporated onto the 3′ end of said sequencing primer then a sequencing reaction byproduct is formed; and
(d) identifying the sequencing reaction byproduct with an ATP generating polypeptide-ATP converting polypeptide fusion protein, thereby determining the nucleotide complementary to the base at said target position.

130. The method of claim 129 wherein the ATP generating polypeptide is selected from the group consisting of ATP sulfurylase, hydrolase and ATP synthase.

131. The method of claim 130 wherein the ATP sulfurylase is a thermostable sulfurylase.

132. The method of claim 130 wherein the ATP sulfurylase is from a thermophile.

133. The method of claim 132 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

134. The method of claim 129 wherein the ATP generating polypeptide and ATP converting polypeptide are from a eukaryote or a prokaryote.

135. The method of claim 134 wherein the eukaryote is selected from the group consisting of animal, plant, fungus and yeast.

136. The method of claim 129 wherein the ATP converting polypeptide is selected from the group consisting of luciferase, ecto-nucleoside diphosphate kinase and ATPase.

137. The method of claim 136 wherein the luciferase is selected from the group consisting of Photinus pyralis, Pyroplorus plagiophihalamus (Coleoptera), Luciola cruciata and Luciola lateralis.

138. The method of claim 129 which further comprises an affinity tag.

139. A method of identifying a base at a target position in a sample DNA sequence comprising:
(a) providing sample DNA disposed within a plurality of cavities on a planar surface, each cavity forming an analyte reaction chamber, wherein the reaction chambers have a center to center spacing of between 5 to 200 μm, said DNA being rendered single stranded either before or after being disposed in the reaction chambers;
(b) providing an extension primer which hybridizes to the sample DNA immediately adjacent to the target position;
(c) subjecting the sample DNA sequence and the extension primer to a polymerase reaction in the presence of a nucleotide triphosphate whereby the nucleotide triphosphate will only become incorporated and release pyrophosphate (PPi) if it is complementary to the base in the target position, said nucleotide triphosphate being added either to separate aliquots of sample-primer mixture or successively to the same sample-primer mixture; and
(d) detecting the release of PPi with an ATP generating polypeptide-ATP converting polypeptide fusion protein to indicate which nucleotide is incorporated.

140. The method of claim 139 wherein the ATP generating polypeptide is selected from the group consisting of ATP sulfurylase, hydrolase and ATP synthase.

141. The method of claim 140 wherein the ATP sulfurylase is a thermostable sulfurylase.

142. The method of claim 140 wherein the ATP sulfurylase is from a thermophile.

143. The method of claim 142 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

144. The method of claim 139 wherein the ATP generating polypeptide and ATP converting polypeptide are from a eukaryote or a prokaryote.

145. The method of claim 144 wherein the eukaryote is selected from the group consisting of animal, plant, fungus and yeast.

146. The method of claim 139 wherein the ATP converting polypeptide is selected from the group consisting of luciferase, ecto-nucleoside diphosphate kinase and ATPase.

147. The method of claim 146 wherein the luciferase is selected from the group consisting of Photinus pyralis, Pyroplorus plagiophihalamus (Coleoptera), Luciola cruciata and Luciola lateralis.

148. The method of claim 139 which further comprises an affinity tag.

149. A method of identifying a base at a target position in a single-stranded sample DNA sequence, the method comprising:
(a) providing an extension primer which hybridizes to sample DNA immediately adjacent to the target position, said sample DNA disposed within a plurality of cavities on a planar surface, each cavity forming an analyte reaction chamber, wherein the reaction chambers have a center to center spacing of between 5 to 200 um, said DNA being rendered single stranded either before or after being disposed in the reaction chambers;
(b) subjecting the sample DNA and extension primer to a polymerase reaction in the presence of a predetermined deoxynucleotide or dideoxynucleotide whereby the deoxynucleotide or dideoxynucleotide will only become incorporated and release pyrophosphate (PPi) if it is complementary to the base in the target position, said predetermined deoxynucleotides or dideoxynucleotides being added either to separate aliquots of sample-primer mixture or successively to the same sample-primer mixture, (c) detecting any release of PPi with an ATP generating polypeptide-ATP converting polypeptide fusion protein to indicate which deoxynucleotide or dideoxynucleotide is incorporated;
characterized in that, the PPi-detection enzyme(s) are included in the polymerase reaction step and in that in place of deoxy- or dideoxy adenosine triphosphate (ATP) a dATP or ddATP analogue is used which is capable of acting as a substrate for a polymerase but incapable of acting as a substrate for a said PPi—detection enzyme.

150. The method of claim 149 wherein the ATP generating polypeptide is selected from the group consisting of ATP sulfurylase, hydrolase and ATP synthase.

151. The method of claim 150 wherein the ATP sulfurylase is a thermostable sulfurylase.

152. The method of claim 150 wherein the ATP sulfurylase is from a thermophile.

153. The method of claim 152 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

154. The fusion protein of claim 149 wherein the ATP generating polypeptide and ATP converting polypeptide are from a eukaryote or a prokaryote.

155. The method of claim 154 wherein the eukaryote is selected from the group consisting of animal, plant, fungus and yeast.

156. The method of claim 149 wherein the ATP converting polypeptide is selected from the group consisting of luciferase, ecto-nucleoside diphosphate kinase and ATPase.

157. The method of claim 156 wherein the luciferase is selected from the group consisting of Photinus pyralis, Pyroplorus plagiophihalamus (Coleoptera), Luciola cruciata and Luciola lateralis.

158. The method of claim 149 which further comprises an affinity tag.

159. A method of determining the base sequence of a plurality of nucleotides on an array, the method comprising:
(a) providing a plurality of sample DNAs, each disposed within a plurality of cavities on a planar surface, each cavity forming an analyte reaction chamber, wherein the reaction chambers have a center to center spacing of between 5 to 200 μm,
(b) converting PPi into light with a an ATP generating polypeptide-ATP converting polypeptide fusion protein;
(c) detecting the light level emitted from a plurality of reaction sites on respective portions of an optically sensitive device;
(d) converting the light impinging upon each of said portions of said optically sensitive device into an electrical signal which is distinguishable from the signals from all of said other regions;
(e) determining a light intensity for each of said discrete regions from the corresponding electrical signal;
(f) recording the variations of said electrical signals with time.

160. The method of claim 159 wherein the ATP generating polypeptide is selected from the group consisting of ATP sulfurylase, hydrolase and ATP synthase.

161. The method of claim 160 wherein the ATP sulfurylase is a thermostable sulfurylase.

162. The method of claim 160 wherein the ATP sulfurylase is from a thermophile.

163. The method of claim 162 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

164. The method of claim 159 wherein the ATP generating polypeptide and ATP converting polypeptide are from a eukaryote or a prokaryote.

165. The method of claim 164 wherein the eukaryote is selected from the group consisting of animal, plant, fungus and yeast.

166. The method of claim 159 wherein the ATP converting polypeptide is selected from the group consisting of luciferase, ecto-nucleoside diphosphate kinase and ATPase.

167. The method of claim 166 wherein the luciferase is selected from the group consisting of Photinus pyralis, Pyroplorus plagiophihalamus (Coleoptera), Luciola cruciata and Luciola lateralis.

168. The method of claim 159 which further comprises an affinity tag.

169. Method for sequencing a nucleic acid, the method comprising:
(a) providing one or more nucleic acid anchor primers;
(b) providing a plurality of single-stranded circular nucleic acid templates disposed within a plurality of cavities on a planar surface, each cavity forming an analyte reaction chamber, wherein the reaction chambers have a center to center spacing of between 5 to 200 μm;
(c) converting PPi into a detectable entity with the use of an ATP generating polypeptide-ATP converting polypeptide fusion protein;
(d) detecting the light level emitted from a plurality of reaction sites on respective portions of an optically sensitive device;
(e) converting the light impinging upon each of said portions of said optically sensitive device into an electrical signal which is distinguishable from the signals from all of said other regions;
(f) determining a light intensity for each of said discrete regions from the corresponding electrical signal;
(g) recording the variations of said electrical signals with time.

170. The method of claim 169 wherein the ATP generating polypeptide is selected from the group consisting of ATP sulfurylase, hydrolase and ATP synthase.

171. The method of claim 170 wherein the ATP sulfurylase is a thermostable sulfurylase.

172. The method of claim 170 wherein the ATP sulfurylase is from a thermophile.

173. The method of claim 172 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

174. The method of claim 169 wherein the ATP generating polypeptide and ATP converting polypeptide are from a eukaryote or a prokaryote.

175. The method of claim 174 wherein the eukaryote is selected from the group consisting of animal, plant, fungus and yeast.

176. The method of claim 169 wherein the ATP converting polypeptide is selected from the group consisting of luciferase, ecto-nucleoside diphosphate kinase and ATPase.

177. The method of claim 176 wherein the luciferase is selected from the group consisting of Photinus pyralis, Pyroplorus plagiophihalamus (Coleoptera), Luciola cruciata and Luciola lateralis.

178. The method of claim 169 which further comprises an affinity tag.

179. A method for sequencing a nucleic acid, the method comprising:
(a) providing at least one nucleic acid anchor primer;
(b) providing a plurality of single-stranded circular nucleic acid templates in an array having at least 400,000 discrete reaction sites;
(c) converting PPi into a detectable entity with an ATP generating polypeptide-ATP converting polypeptide fusion protein;
(d) detecting the light level emitted from a plurality of reaction sites on respective portions of an optically sensitive device;
(e) converting the light impinging upon each of said portions of said optically sensitive device into an electrical signal which is distinguishable from the signals from all of said other regions;
(f) determining a light intensity for each of said discrete regions from the corresponding electrical signal;
(g) recording the variations of said electrical signals with time.

180. The method of claim 179 wherein the ATP generating polypeptide is selected from the group consisting of ATP sulfurylase, hydrolase and ATP synthase.

181. The method of claim 180 wherein the ATP sulfurylase is a thermostable sulfurylase.

182. The method of claim 180 wherein the ATP sulfurylase is from a thermophile.

183. The method of claim 182 wherein the thermophile is a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

184. The method of claim 179 wherein the ATP generating polypeptide and ATP converting polypeptide are from a eukaryote or a prokaryote.

185. The method of claim 184 wherein the eukaryote is selected from the group consisting of animal, plant, fungus and yeast.

186. The method of claim 179 wherein the ATP converting polypeptide is selected from the group consisting of luciferase, ecto-nucleoside diphosphate kinase and ATPase.

187. The method of claim 186 wherein the luciferase is selected from the group consisting of Photinus pyralis, Pyroplorus plagiophihalamus (Coleoptera), Luciola cruciata and Luciola lateralis.

188. The method of claim 179 which further comprises an affinity tag.

189. A kit comprising a sulfurylase-luciferase fusion protein expression vector as claimed in claim 39.

190. An isolated polypeptide comprising an amino acid sequence selected from the group consisting of:
(a) a mature form of an amino acid sequence of SEQ ID NO: 2;
(b) a variant of a mature form of an amino acid sequence of SEQ ID NO: 2;
(c) an amino acid sequence of SEQ ID NO: 2;
(d) a variant of an amino acid sequence of SEQ ID NO: 2, wherein one or more amino acid residues in said variant differs from the amino acid sequence of said mature form, provided that said variant differs in no more than 4% of amino acid residues from said amino acid sequence; and
(e) an amino acid sequence of (a), (b), (c) or (d) further containing one or more conservative amino acid substitutions.

191. The polypeptide of claim 190 wherein said polypeptide comprises the amino acid sequence of a naturally-occurring allelic variant of an amino acid sequence of SEQ ID NO: 2.

192. The polypeptide of claim 190 wherein the amino acid sequence of said variant comprises one or more conservative amino acid substitution.

193. An isolated nucleic acid molecule comprising a nucleic acid sequence encoding a polypeptide comprising an amino acid sequence selected from the group consisting of:
(a) a mature form of an amino acid sequence of SEQ ID NO: 2;
(b) a variant of a mature form of an amino acid sequence of SEQ ID NO: 2, wherein one or more amino acid residues in said variant differs from the amino acid sequence of said mature form, provided that said variant differs in no more than 4% of the amino acid residues from the amino acid sequence of said mature form;
(c) an amino acid sequence of SEQ ID NO: 2;
(d) a variant of an amino acid sequence of SEQ ID NO: 2, wherein one or more amino acid residues in said variant differs from the amino acid sequence of said mature form, provided that said variant differs in no more than 4% of amino acid residues from said amino acid sequence;
(e) a nucleic acid fragment encoding at least a portion of a polypeptide comprising an amino acid sequence of SEQ ID NO: 2, or a variant of said polypeptide, wherein one or more amino acid residues in said variant differs from the amino acid sequence of said mature form, provided that said variant differs in no more than 4% of amino acid residues from said amino acid sequence; and
(f) a nucleic acid molecule comprising the complement of (a), (b), (c), (d) or (e).

194. The nucleic acid molecule of claim 193 wherein the nucleic acid molecule comprises the nucleotide sequence of a naturally-occurring allelic nucleic acid variant.

195. The nucleic acid molecule of claim 193 wherein the nucleic acid molecule encodes a polypeptide comprising the amino acid sequence of a naturally-occurring polypeptide variant.

196. The nucleic acid molecule of claim 193 wherein the nucleic acid molecule comprises nucleotide sequence selected from the group consisting of:
(a) a first nucleotide sequence comprising a coding sequence differing by one or more nucleotide sequences from a coding sequence encoding said amino acid sequence, provided that no more than 11% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

197. A vector comprising the nucleic acid molecule of claim 196.

198. The vector of claim 197, further comprising a promoter operably-linked to said nucleic acid molecule.

199. A cell comprising the vector of claim 197.

200. An antibody that binds immunospecifically to the polypeptide of claim 190.

201. A method for determining the nucleic acid sequence in a template nucleic acid polymer, comprising:
(a) introducing the template nucleic acid polymer into a polymerization environment in which the nucleic acid polymer will act as a template polymer for the synthesis of a complementary nucleic acid polymer when nucleotides are added;
(b) successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released;
(c) separately recovering each of the feedstocks from the polymerization environment; and
(d) measuring the amount of PPi with a thermostable sulfurylase and a luciferase in each of the recovered feedstocks to determine the identity of each nucleotide in the complementary polymer and thus the sequence of the template polymer.

202. The method of claim 201 wherein said thermostable sulfurylase comprises the amino acid sequence of a naturally-occurring allelic variant of an amino acid sequence of SEQ ID NO:2.

203. The method of claim 201 wherein the thermostable sulfurylase is derived from a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

204. The method of claim 201 wherein the thermostable sulfurylase and the luciferase are joined in a fusion protein.

205. The method of claim 201 wherein the thermostable sulfurylase is joined to an affinity tag.

206. A method for sequencing a nucleic acid, the method comprising:
(a) providing one or more nucleic acid anchor primers;
(b) providing a plurality of single-stranded circular nucleic acid templates disposed within a plurality of cavities on a planar surface, each cavity forming an analyte reaction chamber, wherein the reaction chambers have a center to center spacing of between 5 to 200;
(c) annealing an effective amount of the nucleic acid anchor primer to at least one of the single-stranded circular templates to yield a primed anchor primer-circular template complex;
(d) combining the primed anchor primer-circular template complex with a polymerase to form an extended anchor primer covalently linked to multiple copies of a nucleic acid complementary to the circular nucleic acid template;
(e) annealing an effective amount of a sequencing primer to one or more copies of said covalently linked complementary nucleic acid;
(f) extending the sequencing primer with a polymerase and a predetermined nucleotide triphosphate to yield a sequencing product and, if the predetermined nucleotide triphosphate is incorporated onto the 3′ end of said sequencing primer, a sequencing reaction byproduct; and
(g) identifying the sequencing reaction byproduct with the use of a thermostable sulfurylase and a luciferase, thereby determining the sequence of the nucleic acid.

207. The method of claim 206 wherein said thermostable sulfurylase comprises the amino acid sequence of a naturally-occurring allelic variant of an amino acid sequence of SEQ ID NO: 2.

208. The method of claim 206 wherein the thermostable sulfurylase is derived from a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

209. The method of claim 206 wherein the thermostable sulfurylase and the luciferase are joined in a fusion protein.

210. The method of claim 206 wherein the thermostable sulfurylase is joined to an affinity tag.

211. A method for sequencing a nucleic acid, the method comprising:
(a) providing at least one nucleic acid anchor primer;
(b) providing a plurality of single-stranded circular nucleic acid templates in an array having at least 400,000 discrete reaction sites;
(c) annealing a first amount of the nucleic acid anchor primer to at least one of the single-stranded circular templates to yield a primed anchor primer-circular template complex;
(d) combining the primed anchor primer-circular template complex with a polymerase to form an extended anchor primer covalently linked to multiple copies of a nucleic acid complementary to the circular nucleic acid template;
(e) annealing a second amount of a sequencing primer to one or more copies of the covalently linked complementary nucleic acid;
(f) extending the sequencing primer with a polymerase and a predetermined nucleotide triphosphate to yield a sequencing product and, when the predetermined nucleotide triphosphate is incorporated onto the 3′ end of the sequencing primer, to yield a sequencing reaction byproduct; and
(g) identifying the sequencing reaction byproduct with the use of a thermostable sulfurylase and a luciferase, thereby determining the sequence of the nucleic acid at each reaction site that contains a nucleic acid template.

212. The method of claim 211 wherein said thermostable sulfurylase comprises the amino acid sequence of a naturally-occurring allelic variant of an amino acid sequence of SEQ ID NO: 2.

213. The method of claim 211 wherein the thermostable sulfurylase is derived from a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

214. The method of claim 211 wherein the thermostable sulfurylase and the luciferase are joined in a fusion protein.

215. The method of claim 211 wherein the thermostable sulfurylase is joined to an affinity tag.

216. A method of determining the base sequence of a plurality of nucleotides on an array, the method comprising:
(a) providing a plurality of sample DNAs, each disposed within a plurality of cavities on a planar surface, each cavity forming an analyte reaction chamber, wherein the reaction chambers have a center to center spacing of between 5 to 200 μm,
(b) adding an activated nucleotide 5′-triphosphate precursor of one known nitrogenous base to a reaction mixture in each reaction chamber, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates;
(c) detecting whether or not the nucleoside 5′-triphosphate precursor was incorporated into the primer strands through detection of a sequencing byproduct with a thermostable sulfurylase and luciferase, thus indicating that the unpaired nucleotide residue of the template has a nitrogenous base composition that is complementary to that of the incorporated nucleoside 5′-triphosphate precursor; and
(d) sequentially repeating steps (b) and (c), wherein each sequential repetition adds and, detects the incorporation of one type of activated nucleoside 5′-triphosphate precursor of known nitrogenous base composition; and
(e) determining the base sequence of the unpaired nucleotide residues of the template in each reaction chamber from the sequence of incorporation of said nucleoside precursors.

217. The method of claim 216 wherein said thermostable sulfurylase comprises the amino acid sequence of a naturally-occurring allelic variant of an amino acid sequence of SEQ ID NO: 2.

218. The method of claim 216 wherein the thermostable sulfurylase is derived from a thermophilic bacteria selected from the group consisting of Bacillus stearothermophilus, Thermus thermophilus, Bacillus caldolyticus, Bacillus subtilis, Bacillus thermoleovorans, Pyrococcus furiosus, Sulfolobus acidocaldarius, Rhodothermus obamensis, Aquifex aeolicus, Archaeoglobus fulgidus, Aeropyrum pernix, Pyrobaculum aerophilum, Pyrococcus abyssi, Penicillium chrysogenum, Sulfolobus solfataricus and Thermomonospora fusca.

219. The method of claim 216 wherein the thermostable sulfurylase and the luciferase are joined in a fusion protein.

220. The method of claim 216 wherein the thermostable sulfurylase is joined to an affinity tag.


SEQUENCE LISTING (31 sequences)

SEQ ID NO: 1 - DNA, 1247 bp, Bacillus stearothermophilus
gttatgaacatgagtttgagcattccgcatggcggcacattgatcaaccgttggaatccggattacccaatcgatgaagcaacgaaaacgatcgagctgtccaaagccgaactaagcgaccttgagctgatcggcacaggcgcctacagcccgctcaccgggtttttaacgaaagccgattacgatgcggtcgtagaaacgatgcgcctcgctgatggcactgtctggagcattccgatcacgctggcggtgacggaagaaaaagcgagtgaactcactgtcggcgacaaagcgaaactcgtttatggcggcgacgtctacggcgtcattgaaatcgccgatatttaccgcccggataaaacgaaagaagccaagctcgtctataaaaccgatgaactcgctcacccgggcgtgcgcaagctgtttgaaaaaccagatgtgtacgtcggcggagcggttacgctcgtcaaacggaccgacaaaggccagtttgctccgttttatttcgatccggccgaaacgcggaaacgatttgccgaactcggctggaataccgtcgtcggcttccaaacacgcaacccggttcaccgcgcccatgaatacattcaaaaatgcgcgcttgaaatcgtggacggcttgtttttaaacccgctcgtcggcgaaacgaaagcggacgatattccggccgacatccggatggaaagctatcaagtgctgctggaaaactattatccgaaagaccgcgttttcttgggcgtcttccaagctgcgatgcgctatgccggtccgcgcgaagcgattttccatgccatggtgcggaaaaacttcggctgcacgcacttcatcgtcggccgcgaccatgcgggcgtcggcaactattacggcacgtatgatgcgcaaaaaatcttctcgaactttacagccgaagagcttggcattacaccgctctttttcgaacacagcttttattgcacgaaatgcgaaggcatggcatcgacgaaaacatgcccgcacgacgcacaatatcacgttgtcctttctggcacgaaagtccgtgaaatgttgcgtaacggccaagtgccgccgagcacattcagccgtccggaagtggccgccgttttgatcaaagggctgcaagaacgcgaaacggtcaccccgtcgacacgctaaaggaggagcgagatgagcacgaatatcgtttggcatcatacatcggtgacaaaagaagatcgccgccaacgcaacgg

SEQ ID NO: 2 - protein, 386 aa, Bacillus stearothermophilus
MSLSIPHGGTLINRWNPDYPIDEATKTIELSKAELSDLELIGTGAYSPLTGFLTKADYDAVVETMRLADGTVWSIPITLAVTEEKASELTVGDKAKLVYGGDVYGVIEIADIYRPDKTKEAKLVYKTDELAHPGVRKLFEKPDVYVGGAVTLVKRTDKGQFAPFYFDPAETRKRFAELGWNTVVGFQTRNPVHRAHEYIQKCALEIVDGLFLNPLVGETKADDIPADIRMESYQVLLENYYPKDRVFLGVFQAAMRYAGPREAIFHAMVRKNFGCTHFIVGRDHAGVGNYYGTYDAQKIFSNFTAEELGITPLFFEHSFYCTKCEGMASTKTCPHDAQYHVVLSGTKVREMLRNGQVPPSTFSRPEVAAVLIKGLQERETVTPSTR

SEQ ID NO: 3 - DNA, 3519 bp, Escherichia coli
atgcggggttctcatcatcatcatcatcatggtatggctagcatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagggatccgagctcgagatccaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattggcggccgctatgcctgctcctcacggtggtattctacaagacttgattgctagagatgcgttaaagaagaatgaattgttatctgaagcgcaatcttcggacattttagtatggaacttgactcctagacaactatgtgatattgaattgattctaaatggtgggttttctcctctgactgggtttttgaacgaaaacgattactcctctgttgttacagattcgagattagcagacggcacattgtggaccatccctattacattagatgttgatgaagcatttgctaaccaaattaaaccagacacaagaattgcccttttccaagatgatgaaattcctattgctatacttactgtccaggatgtttacaagccaaacaaaactatcgaagccgaaaaagtcttcagaggtgacccagaacatccagccattagctatttatttaacgttgccggtgattattacgtcggcggttctttagaagcgattcaattacctcaacattatgactatccaggtttgcgtaagacacctgcccaactaagacttgaattccaatcaagacaatgggaccgtgtcgtagctttccaaactcgtaatccaatgcatagagcccacagggagttgactgtgagagccgccagagaagctaatgctaaggtgctgatccatccagttgttggactaaccaaaccaggtgatatagaccatcacactcgtgttcgtgtctaccaggaaattattaagcgttatcctaatggtattgctttcttatccctgttgccattagcaatgagaatgagtggtgatagagaagccgtatggcatgctattattagaaagaattatggtgcctcccacttcattgttggtagagaccatgcgggcccaggtaagaactccaagggtgttgatttctacggtccatacgatgctcaagaattggtcgaatcctacaagcatgaactggacattgaagttgttccattcagaatggtcacttatttgccagacgaagaccgttatgctccaattgatcaaattgacaccacaaagacgagaaccttgaacatttcaggtacagagttgagacgccgtttaagagttggtggtgagattcctgaatggttctcatatcctgaagtggttaaaatcctaagagaatccaacccaccaagaccaaaacaaggtttttcaattgttttaggtaattcattaaccgtttctcgtgagcaattatccattgctttgttgtcaacattcttgcaattcggtggtggcaggtattacaagatctttgaacacaataataagacagagttactatctttgattcaagatttcattggttctggtagtggactaattattccaaatcaatgggaagatgacaaggactctgttgttggcaagcaaaacgtttacttattagatacctcaagctcagccgatattcagctagagtcagcggatgaacctatttcacatattgtacaaaaagttgtcctattcttggaagacaatggcttttttgtattttaa

SEQ ID NO: 4 - protein, 1172 aa, Escherichia coli
MRGSHHHHHHGMASMEAPAAAEISGHIVRSPMVGTFYRTPSPDAKAFIEVGQKVNVGDTLCIVEAMKMMNQIEADKSGTVKAILVESGQPVEFDEPLVVIEGSELEIQMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLAAAMPAPHGGILQDLIARDALKKNELLSEAQSSDILVWNLTPRQLCDIELILNGGFSPLTGFLNENDYSSVVTDSRLADGTLWTIPITLDVDEAFANQIKPDTRIALFQDDEIPIAILTVQDVYKPNKTIEAEKVFRGDPEHPAISYLFNVAGDYYVGGSLEAIQLPQHYDYPGLRKTPAQLRLEFQSRQWDRVVAFQTRNPMHRAHRELTVRAAREANAKVLIHPVVGLTKPGDIDHHTRVRVYQEIIKRYPNGIAFLSLLPLAMRMSGDREAVWHAIIRKNYGASHFIVGRDHAGPGKNSKGVDFYGPYDAQELVESYKHELDIEVVPFRMVTYLPDEDRYAPIDQIDTTKTRTLNISGTELRRRLRVGGEIPEWFSYPEVVKILRESNPPRPKQGFSIVLGNSLTVSREQLSIALLSTFLQFGGGRYYKIFEHNNKTELLSLIQDFIGSGSGLIIPNQWEDDKDSVVGKQNVYLLDTSSSADIQLESADEPISHIVQKVVLFLEDNGFFVF

SEQ ID NO: 5 - DNA, 1488 bp, Escherichia coli
atgcggggttctcatcatcatcatcatcatggtatggctagcatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagggatccgagctcgagatctgcagcatgagcgtaagcatcccgcatggcggcacattgatcaaccgttggaatccggattacccaatcgatgaagcaacgaaaacgatcgagctgtccaaagccgaactaagcgaccttgagctgatcggcacaggcgcctacagcccgctcaccgggtttttaacgaaagccgattacgatgcggtcgtagaaacgatgcgcctcgctgatggcactgtctggagcattccgatcacgctggcggtgacggaagaaaaagcgagtgaactcactgtcggcgacaaagcgaaactcgtttatggcggcgacgtctacggcgtcattgaaatcgccgatatttaccgcccggataaaacgaaagaagccaagctcgtctataaaaccgatgaactcgctcacccgggcgtgcgcaagctgtttgaaaaaccagatgtgtacgtcggcggagcggttacgctcgtcaaacggaccgacaaaggccagtttgctccgttttatttcgatccggccgaaacgcggaaacgatttgccgaactcggctggaataccgtcgtcggcttccaaacacgcaacccggttcaccgcgcccatgaatacattcaaaaatgcgcgcttgaaatcgtggacggcttgtttttaaacccgctcgtcggcgaaacgaaagcggacgatattccggccgacatccggatggaaagctatcaagtgctgctggaaaactattatccgaaagaccgcgttttcttgggcgtcttccaagctgcgatgcgctatgccggtccgcgcgaagcgattttccatgccatggtgcggaaaaacttcggctgcacgcacttcatcgtcggccgcgaccatgcgggcgtcggcaactattacggcacgtatgatgcgcaaaaaatcttctcgaactttacagccgaagagcttggcattacaccgctctttttcgaacacagcttttattgcacgaaatgcgaaggcatggcatcgacgaaaacatgcccgcacgacgcacaatatcacgttgtcctttctggcacgaaagtccgtgaaatgttgcgtaacggccaagtgccgccgagcacattcagccgtccggaagtggccgccgttttgatcaaagggctgcaagaacgcgaaacggtcgccccgtcagcgcgctaa

SEQ ID NO: 6 - protein, 495 aa, Escherichia coli
MRGSHHHHHHGMASMEAPAAAEISGHIVRSPMVGTFYRTPSPDAKAFIEVGQKVNVGDTLCIVEAMKMMNQIEADKSGTVKAILVESGQPVEFDEPLVVIEGSELEICSMSVSIPHGGTLINRWNPDYPIDEATKTIELSKAELSDLELIGTGAYSPLTGFLTKADYDAVVETMRLADGTVWSIPITLAVTEEKASELTVGDKAKLVYGGDVYGVIEIADIYRPDKTKEAKLVYKTDELAHPGVRKLFEKPDVYVGGAVTLVKRTDKGQFAPFYFDPAETRKRFAELGWNTVVGFQTRNPVHRAHEYIQKCALEIVDGLFLNPLVGETKADDIPADIRMESYQVLLENYYPKDRVFLGVFQAAMRYAGPREAIFHAMVRKNFGCTHFIVGRDHAGVGNYYGTYDAQKIFSNFTAEELGITPLFFEHSFYCTKCEGMASTKTCPHDAQYHVVLSGTKVREMLRNGQVPPSTFSRPEVAAVLIKGLQERETVAPSAR

SEQ ID NO: 7 - DNA, 45 bp, Artificial Sequence
  Description of Artificial Sequence primer
cccttctgcagcatgagcgtaagcatcccgcatggcggcacattg

SEQ ID NO: 8 - DNA, 47 bp, Artificial Sequence
  Description of Artificial Sequence primer
cccgtaagcttttagcgcgctgacggggcgaccgtttcgcgttcttg

SEQ ID NO: 9 - DNA, 48 bp, Artificial Sequence
  Description of Artificial Sequence primer
ccccctcgagatccaaatggaagacgccaaaaacataaagaaaggccc

SEQ ID NO: 10 - DNA, 45 bp, Artificial Sequence
  Description of Artificial Sequence primer
ccccctcgagatccaaatggctgacaaaaacatcctgtatggccc

SEQ ID NO: 11 - DNA, 67 bp, Artificial Sequence
  Description of Artificial Sequence primer
ttgtagaataccaccgtgaggagcaggcatagcggccgccaatttggactttccgcccttcttggcc

SEQ ID NO: 12 - DNA, 64 bp, Artificial Sequence
  Description of Artificial Sequence primer
ttgtagaataccaccgtgaggagcaggcatagcggccgcaccgttggtgtgtttctcgaacatc

SEQ ID NO: 13 - DNA, 37 bp, Artificial Sequence
  Description of Artificial Sequence primer
gcggccgctatgcctgctcctcacggtggtattctac

SEQ ID NO: 14 - DNA, 49 bp, Artificial Sequence
  Description of Artificial Sequence primer
ccccaagcttttaaaatacaaaaaagccattgtcttccaagaataggac

SEQ ID NO: 15 - DNA, 49 bp, Artificial Sequence
  Description of Artificial Sequence primer
ccccggatccatccaaatgcctgctcctcacggtggtattctacaagac

SEQ ID NO: 16 - DNA, 62 bp, Artificial Sequence
  Description of Artificial Sequence primer
gggcctttctttatgtttttggcgtcttccatagcggccgcaaatacaaaaaagccattgtc

SEQ ID NO: 17 - DNA, 41 bp, Artificial Sequence
  Description of Artificial Sequence primer
gcggccgctatggaagacgccaaaaacataaagaaaggccc

SEQ ID NO: 18 - DNA, 41 bp, Artificial Sequence
  Description of Artificial Sequence primer
ccccccatggttacaatttggactttccgcccttcttggcc

SEQ ID NO: 19 - DNA, 59 bp, Artificial Sequence
  Description of Artificial Sequence primer
gggccatacaggatgtttttgtcagccatagcggccgcaaatacaaaaaagccattgtc

SEQ ID NO: 20 - DNA, 38 bp, Artificial Sequence
  Description of Artificial Sequence primer
gcggccgctatggctgacaaaaacatcctgtatggccc

SEQ ID NO: 21 - DNA, 44 bp, Artificial Sequence
  Description of Artificial Sequence primer
ccccaagcttctaaccgttggtgtgtttctcgaacatctgacgc

SEQ ID NO: 22 - protein, 386 aa, Bacillus stearothermophilus
MSVSIPHGGTLINRWNPDYPLDEATKTIELSKAELSDLELIGTGAYSPLTGFLTKTDYDAVVETMRLSDGTVWSIPVTLAVTEEKAKELAVGDKAKLVYRGDVYGVIEIADIYRPDKTKEAKLVYKTDELAHPGVRKLFEKPDVYVGGEITLVKRTDKGQFAAFYFDPAETRKKFAEFGWNTVVGFQTRNPVHRAHEYIQKCALEIVDGLFLNPLVGETKSDDIPADIRMESYQVLLENYYPKDRVFLGVFQAAMRYAGPREAIFHAMVRKNFGCTHFIVGRDHAGVGNYYGTYDAQKIFLNFTAEELGITPLFFEHSFYCTKCEGMASTKTCPHDAKYHVVLSGTKVREMLRNGQVPPSTFSRPEVAAVLIKGLQERETVAPSAR

SEQ ID NO: 23 - protein, 546 aa, Aquifex aeolicus
MEKIKYLKSIQISQRSVLDLKLLAVGAFTPLDRFMGEEDYRNVVESMRLKSGTLFPIPITLPMEKEIAKDLKEGEWIVLRDPKNVPLAIMRVEEVYKWNLEYEAKNVLGTTDPRHPLVAEMHTWGEYYISGELKVIQLPKYYDFPEYRKTPKQVREEIKSLGLDKIVAFQTRNPMHRVHEELTKRAMEKVGGGLLLHPVVGLTKPGDVDVYTRMRIYKVLYEKYYDKKKTILAFLPLAMRMAGPREALWHGIIRRNYGATHFIVGRDHASPGKDSKGKPFYDPYEAQELFKKYEDEIGIKMVPFEELVYVPELDQYVEINEAKKRNLKYINISGTEIRENFLKQGRKLPEWFTRPEVAEILAETYVPKHKQGFCVWLTGLPCAGKSTIAEILATMLQARGRKVTLLDGDVVRTHLSRGLGFSKEDRITNILRVGFVASEIVKHNGVVICALVSPYRSARNQVRNMMEEGKFIEVFVDAPVEVCEERDVKGLYKKAKEGLIKGFTGVDDPYEPPVAPEVRVDTTKLTPEESALKILEFLKKEGFIKD

SEQ ID NO: 24 - protein, 259 aa, Pyrococcus furiosus
MVSKPHGGKLIRRIAAPRTRERILSEQHEYPRVQIDHGRAIDLENIAHGVYSPLKGFLTREDFESVLDYMRLSDDTPWTIPIVLDVGEPTFEGGDAILLYYENPPIARMHVEDIYTYDKKEFAVKVFKTDDPNHLGVARVYSMGKYLVGGGIELLNELPNPFAKYTLRPVETRILFKERGWKTIVAFQTRNVPHLGHEYVQKAALTFVDGLFINPVLGRKKKGDYKDEVIIKAYYLIMKYCSNTTHHAIMRKTSTSSQT

SEQ ID NO: 25 - protein, 406 aa, Sulfolobus solfataricus
MNLIGHGKVEIVERIKTISDFKELHRIEVKRQLAHEIVSIAYGFLSPLKGFMNYEEVDGVVENMRLPNGVLWPIPLVFDYSQNEKVKEGDTIGITYLGKPLAIMKVKEIFKYDKLKIAEKVYKTKDIKHPGVKRTLSYADAFLAGDVWLVREPQFNKPYSEFWLTPRMHRTVFEKKGWKRVVAFQTRNVPHTGHEYLMKFAWFAANENQKVDEPRTGILVNVVIGEKRVGDYIDEAILLTHDALSKYGYISPKVHLLSFTLWDMRYAGPREALLHAIIRSNLGCTHHVFGRDHAGVGNYYSPYEAHEIFDSINEEDLLIKPIFLRENYYCPRCGSIENEILCDHKDEKQEFSGSLIRSIILDEVKPTKMVMRPEVYDVLMKAAEQYGFGSPFVTEEYLEKRQSILG

SEQ ID NO: 26 - protein, 455 aa, Pyrobaculum aerophilum
MPMPAPLEPHGGRLVYNVIEDRDKAAAMIQGLPSIEIEPTLGPDGSPIRNPYREIMSIAYGFFSPVEGFMTRNEVESILKERRLLNGWLFPFPLIYDVDEEKIKGIKEGDSVLLKLKGKPLAVLNVEEIWRLPDRKELADAVFGTPERNKEVVKKRFDEKHPGWLIYRSMRPMALAGKITVVNPPRFKEPYSRFWMPPRVSREYVEKKGWRIVVAHQTRNVPHIGHEMLMKRAMFVAGGERPGDAVLVNAIIGAKRPGDYVDEAILEGHEALNKAGYFHPDRHVVTMTLWDMRYGNPLESLLHGIIRQNMGATHHMFGRDHAATGDYYDPYATQYLWTRGLPSYGLNEPPHMTDKGLRIKPVNLGEFAYCPKCGEYTYLGMSYEGYKEVALCGHTPERISGSLLRGIIIEGLRPPKVVMRPEVYDVIVKWWRVYGYPYVTDKYLRIKEQELEVEL

SEQ ID NO: 27 - protein, 456 aa, Archaeoglobus fulgidus
MPLIKTPPPHGGKLVERVVKKRDIAEKMIAGCPTYELKPTTLPDGTPIRHVYREIMSVCYGFFSPVEGSMVQNELERVLNERRLLSEWIFPYPILFDISEEDYKALDVKEGDRLLLMLKGQPFATLDIEEVYKIDPVDVATRTFGTPEKNPEVVREPFDDKHPGYVIYKMHNPIILAGKYTIVNEPKFKEPYDRFWFPPSKCREVIKNEKKWRTVIAHQTRNVPHVGHEMLMKCAAYTGDIEPCHGILVNAIIGAKRRGDYPDEAILEGHEAVNKYGYIKPERHMVTFTLWDMRYGNPIESLLHGVIRQNMGCTHHMFGRDHAAVGEYYDMYATQILWSQGIPSFGFEAPPNEVDYGLKIIPQNMAEFWYCPICQEIAYSENCGHTDAKQKFSGSFLRGMVAEGVFPPRVVMRPEVYKQIVKWWKVYNYPFVNRKYLELKNKELEIDLPAMEVPKA

SEQ ID NO: 28 - protein, 573 aa, Penicillium chrysogenum
MANAPHGGVLKDLLARDAPRQAELAAEAESLPAVTLTERQLCDLELIMNGGFSPLEGFMNQADYDRVCEDNRLADGNVFSMPITLDASQEVIDEKKLQAASRITLRDFRDDRNLAILTIDDIYRPDKTKEAKLVFGGDPEHPAIVYLNNTVKEFYIGGKIEAVNKLNHYDYVALRYTPAELRVHFDKLGWSRVVAFQTRNPMHRAHRELTVRAARSRQANVLIHPVVGLTKPGDIDHFTRVRAYQALLPRYPNGMAVLGLLGLAMRMGGPREAIWHAIIRKNHGATHFIVGRDHAGPGSNSKGEDFYGPYDAQHAVEKYKDELGIEVVEFQMVTYLPDTDEYRPVDQVPAGVKTLNISGTELRRRLRSGAHIPEWFSYPEVVKILRESNPPRATQGFTIFLTGYMNSGKDAIARALQVTLNQQGGRSVSLLLGDTVRHELSSELGFTREDRHTNIQRIAFVATELTRAGAAVIAAPIAPYEESRKFARDAVSQAGSFFLVHVATPLEHCEQSDKRGIYAAARRGEIKGFTGVDDPYETPEKADLVVDFSKQSVRSIVHEIILVLESQGFLERQ

SEQ ID NO: 29 - protein, 389 aa, Aeropyrum pernix
MGCSVGLVSRPHGGRLVRRVLSGRRREIFESQYREMPRLEVPLERAIDAEDLARGVFSPLEGFMVEDDYLSVLSRMRLSNDLPWTIPIVLDANREWVLNEGVSAGDDIILTYHGLPIAVLTLEDIYSWDKGLHAEKVFKTRDPNHPGVEATYKRGDILLGGRLELIQGPPNPLERYTLWPVETRVLFKEKGWRTVAAFQTRNVPHLGHEYVQKAALTFVDGLLVHPLAGWKKRGDYRDEVIIRAYEALITHYYPRGVVVLSVLRMNMNYAGPREAVHHAIVRKNFGATHFIVGRDHAGVGSYYGPYEAWEIFREFPDLGITPLFVREAYYCRRCGGMVNEKVCPHGDEYRVRISGTRLREMLGRGERPPEYMMRPEVADAIISHPDPFI

SEQ ID NO: 30 - protein, 511 aa, Saccharomyces cerevisiae
MPAPHGGILQDLIARDALKKNELLSEAQSSDILVWNLTPRQLCDIELILNGGFSPLTGFLNENDYSSVVTDSRLADGTLWTIPITLDVDEAFANQIKPDTRIALFQDDEIPIAILTVQDVYKPNKTIEAEKVFRGDPEHPAISYLFNVAGDYYVGGSLEAIQLPQHYDYPGLRKTPAQLRLEFQSRQWDRVVAFQTRNPMHRAHRELTVRAAREANAKVLIHPVVGLTKPGDIDHHTRVRVYQEIIKRYPNGIAFLSLLPLAMRMSGDREAVWHAIIRKNYGASHFIVGRDHAGPGKNSKGVDFYGPYDAQELVESYKHELDIEVVPFRMVTYLPDEDRYAPIDQIDTTKTRTLNISGTELRRRLRVGGEIPEWFSYPEVVKILRESNPPRPKQGFSIVLGNSLTVSREQLSIALLSTFLQFGGGRYYKIFEHNNKTELLSLIQDFIGSGSGLIIPNQWEDDKDSVVGKQNVYLLDTSSSADIQLESADEPISHIVQKVVLFLEDNGFFVF

SEQ ID NO: 31 - protein, 309 aa, Thermomonospora fusca
MSQVSDAVGRYQLSQLDFLEAEAIFIMREVAAEFERPVLLFSGGKDSVVMLRIAEKAFWPAPIPFPVMHVDTGHNFPEVIEFRDKRVAELGVRLIVASVQDLIDAGKVVEPKGRWASRNRLQTAALLEAIEKYGFDAAFGGARRDEEKARAKERVFSFRDEFGQWDPKNQRPELWNLYNTRVHRGENIRVFPLSNWTELDVWHYIRREGLRLPSIYFAHRRRVFERDGILLPDSPYVTRDEDEEVFEASVRYRTVGDMTCTGAVLSTATTLDEVIAEIAATRITERGQTRADDRGSEAAMEERKREGYF